SEQUENCE LISTING (65 sequences)

SEQ ID NO: 1 - DNA, 59 bp, Artificial Sequence
  Synthetic Sequence
agagagagagagagagagagagagagagagatggaagacgccaaaaacataaagaaagg

SEQ ID NO: 2 - DNA, 19 bp, Artificial Sequence
  Synthetic Sequence
ttaagcgggtcgctgcagg

SEQ ID NO: 3 - DNA, 54 bp, Artificial Sequence
  Synthetic Sequence
acacacacacacacacacacacacacacacatggaagacgccaaaaacataaag

SEQ ID NO: 4 - DNA, 54 bp, Artificial Sequence
  Synthetic Sequence
gcagaccacgtatgtggtcgatgccatggaagacgccaaaaacataaagaaagg

SEQ ID NO: 5 - DNA, 54 bp, Artificial Sequence
  Synthetic Sequence
gcacaccacgtatgtggtcgatgccatggaagacgccaaaaacataaagaaagg

SEQ ID NO: 6 - DNA, 54 bp, Artificial Sequence
  Synthetic Sequence
gcacaacacgtatgtggtcgatgccatggaagacgccaaaaacataaagaaagg

SEQ ID NO: 7 - DNA, 29 bp, Artificial Sequence
  Synthetic Sequence
ggagaggacgtatgtggtcgatgccatgg

SEQ ID NO: 8 - DNA, 29 bp, Artificial Sequence
  Synthetic Sequence
atggaagacgccaaaaacataaagaaagg

SEQ ID NO: 9 - DNA, 33 bp, Artificial Sequence
  Synthetic Sequence
gatgaaggttttaatcttcatcttaagcgggtc

SEQ ID NO: 10 - DNA, 28 bp, Artificial Sequence
  Synthetic Sequence
agagatggaagacgccaaaaacataaag

SEQ ID NO: 11 - DNA, 35 bp, Artificial Sequence
  Synthetic Sequence
tctctgatgaaggttttaatcttcatcttaagcgg

SEQ ID NO: 12 - DNA, 59 bp, Artificial Sequence
  Synthetic Sequence
ccctgcagcgacccgcttaaacttaagtaccttatctatctacagatagaaaagttgct

SEQ ID NO: 13 - DNA, 49 bp, Artificial Sequence
  Synthetic Sequence
atgtttttggcgtcttccattaggaacagttactggtttccacaaagac

SEQ ID NO: 14 - DNA, 48 bp, Artificial Sequence
  Synthetic Sequence
gaaaccagtaactgttcctaatggaagacgccaaaaacataaagaaag

SEQ ID NO: 15 - DNA, 39 bp, Artificial Sequence
  Synthetic Sequence
gatagataaggtacttaagtttaagcgggtcgctgcagg

SEQ ID NO: 16 - DNA, 53 bp, Artificial Sequence
  Synthetic Sequence
ccctgcagcgacccgcttaagatttaagtgaatagcttggctatctcacttcc

SEQ ID NO: 17 - DNA, 40 bp, Artificial Sequence
  Synthetic Sequence
atgtttttggcgtcttccatgatgtgccccgaattgccac

SEQ ID NO: 18 - DNA, 48 bp, Artificial Sequence
  Synthetic Sequence
gtggcaattcggggcacatcatggaagacgccaaaaacataaagaaag

SEQ ID NO: 19 - DNA, 41 bp, Artificial Sequence
  Synthetic Sequence
ccaagctattcacttaaatcttaagcgggtcgctgcagggt

SEQ ID NO: 20 - DNA, 66 bp, Artificial Sequence
  Synthetic Sequence
ccctgcagcgacccgcttaagatgaagattaagaaaaaggtaatctttcgattatctttaatcttc

SEQ ID NO: 21 - DNA, 54 bp, Artificial Sequence
  Synthetic Sequence
atgtttttggcgtcttccatttaaaagttgtctccactcactgtttaaatttct

SEQ ID NO: 22 - DNA, 48 bp, Artificial Sequence
  Synthetic Sequence
tgagtggagacaacttttaaatggaagacgccaaaaacataaagaaag

SEQ ID NO: 23 - DNA, 39 bp, Artificial Sequence
  Synthetic Sequence
cctttttcttaatcttcatcttaagcgggtcgctgcagg

SEQ ID NO: 24 - DNA, 52 bp, Artificial Sequence
  Synthetic Sequence
ccctgcagcgacccgcttaagatgaagattaaaaccttcatcatccttacgt

SEQ ID NO: 25 - DNA, 50 bp, Artificial Sequence
  Synthetic Sequence
atgtttttggcgtcttccatcttgttagaccagcttttcttttacattga

SEQ ID NO: 26 - DNA, 48 bp, Artificial Sequence
  Synthetic Sequence
agaaaagctggtctaacaagatggaagacgccaaaaacataaagaaag

SEQ ID NO: 27 - DNA, 39 bp, Artificial Sequence
  Synthetic Sequence
tgaaggttttaatcttcatcttaagcgggtcgctgcagg

SEQ ID NO: 28 - DNA, 19 bp, Artificial Sequence
  Synthetic Sequence
aagccgtccgccgttatag

SEQ ID NO: 29 - DNA, 19 bp, Artificial Sequence
  Synthetic Sequence
caaatcggcatgccctaaa

SEQ ID NO: 30 - DNA, 20 bp, Artificial Sequence
  Synthetic Sequence
cggtgttaggcgcaagaatt

SEQ ID NO: 31 - DNA, 20 bp, Artificial Sequence
  Synthetic Sequence
aggagcacgggagtcaggtt

SEQ ID NO: 32 - DNA, 21 bp, Artificial Sequence
  Synthetic Sequence
ctgctcctcctgttcgacagt

SEQ ID NO: 33 - DNA, 20 bp, Artificial Sequence
  Synthetic Sequence
ccgttgactccgaccttcac

SEQ ID NO: 34 - DNA, 25 bp, Artificial Sequence
  Synthetic Sequence
gcagaccacgtatgtggtcgatgcc

SEQ ID NO: 35 - DNA, 25 bp, Artificial Sequence
  Synthetic Sequence
ggagaggacgtatgtggtcgatgcc

SEQ ID NO: 36 - DNA, 53 bp, Artificial Sequence
  Synthetic Sequence
ccctgcagcgacccgcttaaattaaaggtttataccttcccaggtaacaaacc

SEQ ID NO: 37 - DNA, 40 bp, Artificial Sequence
  Synthetic Sequence
atgtttttggcgtcttccatcttacctttcggtcacaccc

SEQ ID NO: 38 - DNA, 48 bp, Artificial Sequence
  Synthetic Sequence
gggtgtgaccgaaaggtaagatggaagacgccaaaaacataaagaaag

SEQ ID NO: 39 - DNA, 44 bp, Artificial Sequence
  Synthetic Sequence
ggaaggtataaacctttaatttaagcgggtcgctgcagggtcgc

SEQ ID NO: 40 - DNA, 292 bp, Human coronavirus
acttaagtaccttatctatctacagatagaaaagttgctttttagactttgtgtctacttttctcaactaaacgaaatttttgctatggccggcatctttgatgctggagtcgtagtgtaattgaaatttcatttgggttgcaacagtttggaagcaagtgctgtgtgtcctagtctaagggtttcgtgttccgtcacgagattccattctacaaacgccttactcgaggttccgtctcgtgtttgtgtggaagcaaagttctgtctttgtggaaaccagtaactgttccta

SEQ ID NO: 41 - RNA, 292 nt, Human coronavirus
acuuaaguaccuuaucuaucuacagauagaaaaguugcuuuuuagacuuugugucuacuuuucucaacuaaacgaaauuuuugcuauggccggcaucuuugaugcuggagucguaguguaauugaaauuucauuuggguugcaacaguuuggaagcaagugcuguguguccuagucuaaggguuucguguuccgucacgagauuccauucuacaaacgccuuacucgagguuccgucucguguuuguguggaagcaaaguucugucuuuguggaaaccaguaacuguuccua

SEQ ID NO: 42 - DNA, 278 bp, Human coronavirus
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatc

SEQ ID NO: 43 - RNA, 278 nt, Human coronavirus
gauuuaagugaauagcuuggcuaucucacuuccccucguucucuugcagaacuuugauuuuaacgaacuuaaauaaaagcccuguuguuuagcguaucguugcacuugucuggugggauuguggcauuaauuugccugcucaucuaggcaguggacauaugcucaacacuggguauaauucuaauugaauacuauuuuucaguuagagcgucgugucucuuguacgucucggucacaauacacgguuucguccggugcguggcaauucggggcacauc

SEQ ID NO: 44 - DNA, 265 bp, Human coronavirus
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 45 - RNA, 265 nt, Human coronavirus
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaag

SEQ ID NO: 46 - DNA, 50 bp, Artificial Sequence
  Synthetic Sequence
acatttgcttctgacacaactgtgttcactagcaacctcaaacagacacc

SEQ ID NO: 47 - RNA, 50 nt, Artificial Sequence
  Synthetic Sequence
acauuugcuucugacacaacuguguucacuagcaaccucaaacagacacc

SEQ ID NO: 48 - DNA, 30 bp, Artificial Sequence
  Synthetic Sequence
agagagagagagagagagagagagagagag

SEQ ID NO: 49 - DNA, 30 bp, Artificial Sequence
  Synthetic Sequence
acacacacacacacacacacacacacacac

SEQ ID NO: 50 - DNA, 30 bp, Artificial Sequence
  Synthetic Sequence
agagacacacacacacacacacacacacac

SEQ ID NO: 51 - DNA, 30 bp, Artificial Sequence
  Synthetic Sequence
agagagagagacacacacacacacacacac

SEQ ID NO: 52 - DNA, 30 bp, Artificial Sequence
  Synthetic Sequence
agagagagagagagacacacacacacacac

SEQ ID NO: 53 - DNA, 30 bp, Artificial Sequence
  Synthetic Sequence
agagagagagagagagagagacacacacac

SEQ ID NO: 54 - DNA, 30 bp, Artificial Sequence
  Synthetic Sequence
acacacacacacagagacacacacacacac

SEQ ID NO: 55 - DNA, 30 bp, Artificial Sequence
  Synthetic Sequence
acacacacacagagagagagacacacacac

SEQ ID NO: 56 - skipped

SEQ ID NO: 57 - DNA, 30 bp, Artificial Sequence
  Synthetic Sequence
acacagagagagagagagagagagacacac

SEQ ID NO: 58 - DNA, 25 bp, Artificial Sequence
  Synthetic Sequence
gcagaccacgtatgtggtcgatgcc

SEQ ID NO: 59 - DNA, 25 bp, Artificial Sequence
  Synthetic Sequence
gcacaccacgtatgtggtcgatgcc

SEQ ID NO: 60 - DNA, 25 bp, Artificial Sequence
  Synthetic Sequence
gcacaacacgtatgtggtcgatgcc

SEQ ID NO: 61 - DNA, 25 bp, Artificial Sequence
  Synthetic Sequence
ggagaggacgtatgtggtcgatgcc

SEQ ID NO: 62 - DNA, 221 bp, Artificial Sequence
  Synthetic Sequence
gatgaagattaagaaaaaggtaatctttcgattatctttaatcttcatccttgattctacaatcatgacagttgtctttagtgacaagggaaagaagcctttttattaagttgtaataatcagatctgcgaaccggtagagtttagttgcaacctaacacacataaagcattggtcaaaaagtcaatagaaatttaaacagtgagtggagacaacttttaa

SEQ ID NO: 63 - DNA, 97 bp, Artificial Sequence
  Synthetic Sequence
gatgaagattaaaaccttcatcatccttacgtcaattgaattctctagcactcgaagcttattgtcttcaatgtaaaagaaaagctggtctaacaag

SEQ ID NO: 64 - RNA, 22 nt, Artificial Sequence
  Synthetic Sequence
gaugaagauuaaaaccuucauc

SEQ ID NO: 65 - RNA, 32 nt, Artificial Sequence
  Synthetic Sequence
gaugaagauuaaaaccuucaucagagagagag

(19) United States
(12) Patent Application Publication
(10) Pub. No.: US 2025/0345307 A1
(43) Pub. Date: Nov. 13, 2025

(54) SYNTHETIC ROCAGLATES WITH BROAD-SPECTRUM ANTIVIRAL ACTIVITIES AND USES THEREOF

(71) Applicants: Memorial Sloan Kettering Cancer Center, NEW YORK, NY (US); Philipps-Universität Marburg, Marburg (DE)

(72) Inventors: HANS-GUIDO WENDEL, New York, NY (US); ARNOLD GRUNWELLER, ARNOLD, Marburg (DE)

(73) Assignees: Memorial Sloan Kettering Cancer Center, NEW YORK, NY (US); Philipps-Universität Marburg, Marburg (DE)

(21) Appl. No.: 17/822,043

(22) PCT Filed: Feb. 23, 2021
(86) PCT No.: PCT/US2021/019295

§ 371 Date: Aug. 24, 2022

Related U.S. Application Data
(60) Provisional application No. 62/980,942, filed on Feb. 24, 2020.

(51) Int. Cl. A61K 31/343; A61P 31/14

(52) U.S. Cl. CPC A61K 31/343; A61P 31/14

(57) ABSTRACT

Described herein are compositions, uses thereof, and methods for treating a viral infection in a host cell or organism infected by the virus, such as coronaviruses (e.g., severe acute respiratory syndrome coronavirus [SARS-CoV], severe acute respiratory syndrome coronavirus 2 [SARS-CoV-2, the virus and its mutant forms that cause COVID-19], Middle East respiratory syndrome coronavirus [MERS-CoV]), Zika virus, Lassa virus, Crimean Congo hemorrhagic fever virus, hepatitis E virus, and other RNA viruses. Also described herein are synthetic rocaglate compositions, uses thereof, and methods for reducing or inhibiting translation initiation of a messenger ribonucleic acid (mRNA) of a virus in a host cell or organism infected by the virus.

### CROSS-REFERENCE TO RELATED APPLICATION

This application claims priority to International Application PCT/US2021/019295, filed Feb. 23, 2021, which claims priority to U.S. Provisional Patent Application 62/980,943, filed Feb. 24, 2020, and which are incorporated by reference herein in their entirety.

### GOVERNMENT INTERESTS

This invention was made with government support under CA207217 and CA008748 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 27, 2024, is named “P-593548-US_SQL_ST.25_27FEB24”, and is 14,707 bytes in size.

### FIELD OF INTEREST

This disclosure relates to synthetic rocaglate compositions, uses thereof, and methods for treating a viral infection in a host cell or organism infected by the virus, such as coronaviruses (e.g., severe acute respiratory syndrome coronavirus [SARS-CoV], severe acute respiratory syndrome coronavirus 2 [SARS-CoV-2, the virus and its mutant forms that cause COVID-19], Middle East respiratory syndrome coronavirus [MERS-CoV]), Zika virus, Lassa virus, Crimean Congo hemorrhagic fever virus, and hepatitis E virus, and other RNA viruses. Also disclosed are synthetic rocaglate compositions, uses thereof, and methods for reducing or inhibiting translation initiation of a messenger ribonucleic acid (mRNA) of a virus in a host cell or organism infected by the virus.

### BACKGROUND OF THE INVENTION

Rocaglates, a class of natural compounds isolated from plants of the genus Aglaia in the mahogany family (Meliaceae), are potent inhibitors of translation initiation. They are proposed to form stable stacking interactions with polypurine sequences in the 5′-UTR of selected mRNAs thereby clamping the RNA substrate onto eIF4A causing the inhibition of the translation initiation complex. The DEAD-box RNA helicase eIF4A, which is part of the heterotrimeric translation initiation complex eIF4F, unwinds RNA secondary structures in 5′-untranslated regions (5′-UTRs) of selected mRNAs to enable binding of the 43S preinitiation complex (PIC). In cells, eIF4A has a critical role in the translation of protooncogenic mRNAs with complex structured 5′-UTRs. Viral RNAs also contain highly structured 5′-UTRs, suggesting that viral protein synthesis may also be eIF4A-dependent.

In this regard, the specific eIF4A inhibitor Silvestrol, a plant-derived rocaglate, has broad-spectrum antiviral activity at non-cytotoxic concentrations in a low nanomolar range. Silvestrol inhibits the replication of RNA viruses representing different virus families, like Ebola- (EBOV), Corona- (CoV), Zika- (ZIKV), Chikungunya- (CHIKV), and hepatitis E (HEV) viruses. Since 2000, there have been three documented cases of a coronavirus outbreak of zoonotic origin to reach epidemic or pandemic scale. These outbreaks have been caused by three severe viruses of the Coronaviridae family: Severe acute respiratory syndrome coronavirus (SARS-CoV or SARS-CoV-1) in 2002-2003; Middle East respiratory syndrome (MERS)-related coronavirus (MERS-CoV) in 2012-2013 (which is still ongoing); and severe acute respiratory syndrome coronavirus 2 (SARS-CoV-2), a positive-sense, single-stranded RNA coronavirus of the genus Betacoronavirus in the subgenus Sarbecovirus, which is of zoonotic origin, causes a potentially severe respiratory disease with varying symptoms referred to as coronavirus disease 2019 (COVID-19) and is responsible for a pandemic starting in early 2020. Notably, Silvestrol showed good bioavailability, in vitro, ex vivo and in vivo antiviral activity and low cytotoxicity in primary cells. However, synthesis of Silvestrol is sophisticated, difficult, and time-consuming, thus hampering its prospects for further antiviral clinical development.

It would be desirable to have alternative strategies utilizing additional compositions and methods for inhibiting the replication of pathogenic RNA viruses, including coronaviruses. It would also be desirable to have compositions and methods for treating or preventing human and other animal infections by RNA viruses, including coronaviruses.

### SUMMARY OF THE INVENTION

The compositions and methods provided herein are directed to inhibiting the replication of RNA viruses, including coronaviruses, and to treating or preventing human or other animal infections by RNA viruses, including coronaviruses.

Disclosed herein are methods of treating a viral infection in a host cell or organism infected by the virus, the methods comprising administering to the cell or organism a therapeutically effective amount of a pharmaceutical composition comprising CR-31-B or a pharmaceutically acceptable salt thereof.

In some embodiments, the CR-31-B comprises a racemic mixture of:
i. a CR-31-B (−) enantiomer having the formula

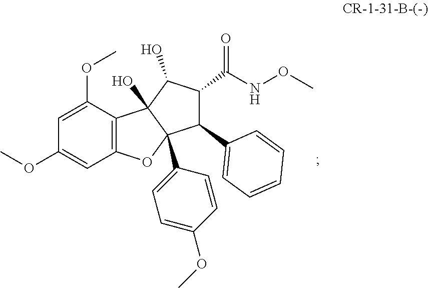

and
ii. a CR-31-B (+) enantiomer having the formula

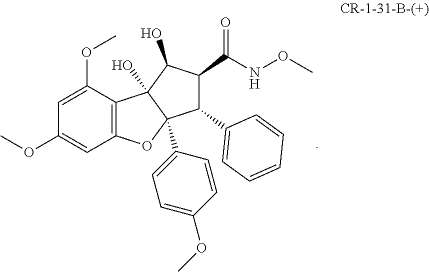

In some embodiments, the CR-31-B comprises at least 50% CR-31-B (−) enantiomer. In some embodiments, the CR-31-B is a CR-31-B (−) enantiomer. In some embodiments, the CR-31-B reduces or inhibits a eukaryotic initiation factor 4A (eIF4A) activity.

Also disclosed herein are methods for reducing or inhibiting translation initiation of a messenger ribonucleic acid (mRNA) of a virus in a host cell or organism infected by the virus, the methods comprising administering to the cell or organism a therapeutically effective amount of a pharmaceutical composition comprising CR-31-B or a pharmaceutically acceptable salt thereof.

Also disclosed herein are uses of a synthetic rocaglate composition for reducing or inhibiting translation initiation of a messenger ribonucleic acid (mRNA) of a virus in a host cell or organism infected by the virus, the synthetic rocaglate composition comprising a therapeutically effective amount of CR-31-B or a pharmaceutically acceptable salt thereof.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic depicting a comparison between the chemical structures of the rocaglates Silvestrol, Episilvestrol, CR-31-B (−), CR-31-B (+), and rocaglamide A (RocA). The characteristic cyclopenta[b]benzofurane structure found in all rocaglates is indicated in red in the Silvestrol structure. The dioxane moiety that is only found in Silvestrol and Episilvestrol is shown on the left side in black. Silvestrol and Episilvestrol are enantiomers that differ only in one —OH group in the dioxan ring (marked with a circle: (R) for Silvestrol and (S) for Episilvestrol). Both have comparable effects as antiviral compounds. The blue ring indicates the variable region in rocaglates.

FIGS. 2A-2C depict antiviral activities of the synthetic rocaglate CR-31-B (−) against coronaviruses HCoV-229E and MERS-CoV. FIG. 2A shows photographs of the results of Western blot analysis of HCoV-229E N protein accumulation (top panel) in cells treated with different enantiomers of CR-31-B (CR-31-B (−), left; CR-31-B (+), right). β-Actin (beta-actin; lower panel) was used as a loading control. FIG. 2B is a graph depicting total (genomic and subgenomic) viral RNA produced in HCoV-229E-infected MRC-5 cells treated with the two enantiomers of CR-31-B. Relative changes in viral RNA levels were determined by RT-qPCR. The data were normalized to infected but untreated cells as well as GAPDH mRNA using the comparative ΔΔCt (delta-delta-Ct) method. FIG. 2C shows graphs of HCoV-229E (left) and MERS-CoV (right) titers in supernatants collected from infected MRC-5 cells (MOI=0.1) at 24 hpi. Cells were treated with CR-31-B (−) as indicated. Data from three independent experiments were used to calculate half maximal effective concentration (EC₅₀) values (2.88 nM for HCoV-229E and 1.87 nM for MERS-CoV).

FIG. 3 is a series of photographs depicting immunofluorescence analysis to visualize the effects of CR-31-B (−) on viral dsRNA (center column) and nonstructural protein 8 (nsp8; left column) accumulation in HCoV-229E-infected MRC-5 cells. Cells were infected with an MOI of 1 and incubated with the indicated CR-31-B (−) concentrations: 10 nM CR-31-B (−) (top row); 0.1 nM CR-31-B (−) (middle row); and 0 nM CR-31-B (−) (control cells treated with DMSO; bottom row). Cells were fixed at 24 hpi and analyzed by confocal laser-scanning microscopy using antibodies specific for dsRNA (red) and nsp8 (green). The images were also merged as shown (right column).

FIGS. 4A-4C show a series of graphs depicting a comparison of CR-31-B (−) vs. CR-31-B (+) with respect to reduction of viral titer and cytotoxicity. FIG. 4A shows bar graphs demonstrating CR-31-B (−) inhibits the production of infectious virus progeny of HCoV-229E (left) and MERS-CoV (right) at low nanomolar concentrations.

FIG. 4B shows graphs demonstrating that treatment of MRC-5 cells for 24 h with CR-31-B (−) and CR-31-B (+) caused no major cytotoxicity at concentrations of up to 5 μM (micromolar) measured via MIT assay. FIG. 4C shows graphs and data demonstrating CC₅₀ values determined via ATPlite assay for MRC-5 cells incubated with CR-31-B (−) or CR-31-B (+) for 24, 48 or 72 h as indicated.

FIG. 5 is a graph depicting CC₅₀ values were determined for a range of human skin carcinoma and liver carcinoma cell lines treated with racemic (+/−) CR-31-B. For comparison, the CC₅₀ value of (+/−) CR-31-B was determined using primary human dermal fibroblasts (HDF). Data is representative of four experimental replicates. The average CC₅₀ across the two biological replicates is plotted.

FIGS. 6A-6C demonstrate a comparison of antiviral effects of CR-31-B (−) vs. Silvestrol using human bronchial epithelial cells infected with HCoV-229E. FIG. 6A is a schematic depicting the method used. Human bronchial epithelial cells were cultivated and differentiated at an air liquid interface into different airway epithelial cell types (basal, ciliated, clara, and goblet cells) and used to assess antiviral effects of the respective compounds. FIG. 6B and FIG. 6C show graphs comparing the effects of treating cells from Donor 1 and Donor 2, respectively. In both instances, HCoV-229E titers in cell culture supernatants were collected at the indicated time points p.i. (h, hours). Cells obtained from the two different donors were infected and treated with Silvestrol (100/10 nM, left), CR-31-B (−) (100/10 nM; right), or CR-31-B (+) (100 nM; right), respectively.

FIGS. 7A-7B are graphs demonstrating the potent antiviral activity of CR-31-B (−) and Silvestrol against the Zika virus (Uganda strain 976) in A549 cells. Cell were infected using a MOI of 0.1 for 16 hours and simultaneously treated with compounds in the concentrations as shown. In FIG. 7A, intracellular RNA was obtained, and after reverse transcription, the ZIKV genomes were quantified by qRT-PCR to determine the EC₅₀. EC₅₀ values of both compounds are approximately 1 nM (EC₅₀ [Silvestrol]=1.08 nM; EC₅₀ [CR-31-B (−)]—1.13 nM). In FIG. 7B, cell viability of A549 cells was determined using the PRESTOBLUE™ cell viability agent (THERMOFISHER SCIENTIFIC™) after treatment with the compounds in their respective concentrations for 72 hours. CC₅₀ values are 9.42 nM for Silvestrol and 19.3 for CR-31-B (−).

FIGS. 8A-8C are graphs showing that CR-31-B (−) and Silvestrol inhibit LASV (left column) and CCHFV (right column) replication in primary murine hepatocytes with comparable efficiencies in a concentration range between 20 and 50 nM.

FIG. 8A demonstrates potent antiviral activity of CR-31-B (−) against LASV and CCHFV without cytotoxicity in murine hepatocytes. FIG. 8B shows no antiviral effects of CR-31-B (+) up to a concentration of 5 μM. FIG. 8C demonstrates potent antiviral activity of Silvestrol against LASV and CCHFV without cytotoxicity in murine hepatocytes.

FIG. 9 shows a graph demonstrating that CR-31-B (−) and Silvestrol reduce the levels of extracellular HEV RNA at low nanomolar concentrations. The graph depicts qRT-PCR measurement of extracellular HEV RNA of CR-31-B (+) (left), CR-31-B (−) (center), and Silvestrol (left) treated, persistently HEV infected cells. All data are referred to the DMSO control.

FIG. 10 is a graph showing analysis of cytotoxicity of CR-31-B (+), CR-31-B (−), and Silvestrol in persistently HEV-infected A549 cells after treatment with the indicated compound for 72 h. At a concentration of 2 nM, both CR-31-B enantiomers had no major cytotoxic effects, whereas the natural rocaglate Silvestrol reduced the cell viability by approximately 30%. At an increased concentration of 10 nM, both CR-31-B (−) and Silvestrol caused a reduction of cell viability by approximately 40%.

FIG. 11 is a schematic of the dual luciferase assay used to analyze the sensitivity of viral 5′-UTRs towards eIF4A inhibition.

FIG. 12 is a graph showing the effects of 5 and 10 nM (nanomolar) Silvestrol on the translation efficiency of reporter gene expression constructs containing different 5′-untranslated regions (5′-UTRs) in the context of the following 5′-UTRs: (AC)₁₅, polyAC-5′-(AG)_{2.5}, polyAC-5′-(AG)₅, polyAC-5′-(AG)_{7.5}, polyAC-5′-(AG)₁₀, poly AC-mid(AG)_{2.5}, polyAC-mid(AG)₅, polyAC-mid-(AG)_{7.5}, polyAC-mid(AG)₁₀, and (AG)₁₅. Results were normalized to dimethyl sulfoxide (DMSO).

FIGS. 13A-13B depict a comparison of the inhibitory effects of CR-31-B (−) and Silvestrol on reporter gene expression constructs containing different viral 5′-UTRs. FIG. 13A has a graph (above) showing the effects of 5 and 10 nM Silvestrol or CR-31-B (−) on the translation efficiency of reporter gene expression in the context of 5′-UTRs from coronaviruses HCoV-229E and MERS-CoV, as well as EBOV VP30 and VP35. The VP35 5′-terminal hairpin and the VP35 hairpin with (AG)₅ extensions were also analyzed. Results were normalized to DMSO. The predicted RNA secondary structures of the indicated 5′UTRs are shown (below). Asterisks mark the positions of purines as part of the polypurine stretch in the VP35 hairpin. FIG. 13B has a graph (above) showing the analysis of the sensitivity of the 5′-UTR of HEV and derivatives thereof against 5 and 10 nM Silvestrol and CR-31-B (−) treatment in a dual luciferase assay. (AG)₁₅ and (AC)₁₅ sequences were used as positive and negative controls, respectively. Predicted RNA secondary structures of the HEV 5′-UTRs are shown (below). The reporter gene expression data were normalized to the transfection efficiencies and the corresponding DMSO controls. Blue circles indicate the mutated nucleotides in HEVgt3c-G4C and HEVgt3c-G4CC6A. Asterisks mark the positions of purines as part of the polypurine stretch in HEVgt3c-Purine. Standard errors of the mean of at least three independent experiments are shown. MFE=minimal free energy (kcal/mol).

FIG. 14 is a schematic depicting a model of the activity of this class of drugs (rocaglates) on eIF4A and its ability to block unwinding of RNA with secondary structures by Silvestrol or CR-31-B (−) as exemplary inhibitors of eIF4A, which is shown as a model of the surface of human eIF4A with a bound polyAG sequence. Interaction of eIF4A with RNA unwinds the viral mRNA 5′-UTR hairpin structure upstream of the polyAG sequence to enable translation initiation at the AUG start codon downstream of the polyAG sequence, as depicted in the schematic. This general viral mRNA structure is common to HCoV-229E, MERS-CoV, LASV, CCHFV, ZIKV, and HEV, as well as other RNA viruses, although there are differences in the nature of the 5′-UTRs.

FIGS. 15A-15E are models for the possible binding mode of Silvestrol and RNA clamping using structure-based comparative modeling of RocA or Silvestrol onto a 10-mer polyAG RNA bound to the surface of human eIF4A. FIG. 15A depicts the surface of human eIF4A with a bound polyAG 10-mer (adapted from Iwasaki et al. [2019] Mol. Cell 73: 738-748 e9) for comparison. FIGS. 15B-15E zoom in to show the binding region of RocA and Silvestrol. The dioxane moiety of Silvestrol is able to cross the RNA stretch to make additional contacts with proximal positioned arginine residues in eIF4A. The PYMOL™ (SCHROEDINGER®) molecular graphics system was used for graphical illustration. eIF4A: grey; RNA: green; RocA: purple; Silvestrol: cyan.

FIGS. 16A-16B depict models for the possible binding mechanism of CR-31-B (−). FIG. 16A (upper panel) is a model for the predicted binding mode of CR-31-B (−) on a human eIF4A-RNA complex (red depicts negative charges; blue depicts positive charges; white depicts neutral amino acids with no charge or polar groups in the side chain; gold depicts the RNA structure). Binding of CR-31-B (−) leads to RNA clamping shown by structure-based comparative modeling. FIG. 16A (lower panel) is a schematic depicting SARS-CoV-2 infected cells, the predicted secondary structure of the SARS-CoV-2 5′-UTR with the 5′-cap bound translation initiation complex eIF4F (consisting of the cap-binding protein eIF4E, the bridging protein eIF4G and the DEAD-box RNA helicase eIF4A). RNA clamping of CR-31-B (−) blocks translation and strongly reduces viral protein synthesis and as a consequence viral replication. FIG. 16B is a schematic depicting the RNA sequence (SEQ ID NO: 45; see Table 6) and predicted secondary structure of the SARS-CoV-2 5′UTR (identical with Group IIa). RNA secondary structures that form stem loops are abbreviated with SL. The sequence ends directly before the start codon AUG (marked in yellow) ( . . . AAG AUG . . . ) and is located between SL5c and SL5.

FIG. 17 is a graph depicting the effect of CR-31-B (−) on reporter gene expression from constructs containing different 5′-UTRs using the dual luciferase reporter construct of FIG. 11 to determine eIF4A-dependent translation of coronavirus 5′-UTRs. The effects of 5 and 10 nM CR-31-B (−) on reporter gene expression are shown in the context of 5′-UTRs from three human coronaviruses, HCoV-229E, MERS-CoV, and SARS-CoV-2. The 5′-UTR of the human beta(β)-globin mRNA and the unstructured (AC)₁₅ sequence served as negative controls, while the (AG)₁₅ polypurine sequence served as a positive control. Experiments were performed with at least three independent biological replicates (Müller et al. [2018] Antiviral Res. 150: 123-129; Müller et al. [2020] Antiviral Res. 175: 104706).

FIGS. 18A-18D are graphs, a Western blot, and an immunofluorescence depicting dose-dependent antiviral activity of the synthetic rocaglate CR-31-B (−) in SARS-CoV-2 infected Vero E6 cells. FIG. 18A is a graph depicting an MTT (3-(4,5-dimethylthiazol-2-yl)-2,5-diphenyltetrazolium bromide) assay of Vero E6 cells treated for 24 h with the indicated CR-31-B (−) concentrations. Cell viability (compared to that of untreated cells) was determined by MTT assay (n=8) as described herein (see also Miller et al. [2018] Antiviral Res. 150: 123-129). FIG. 18B shows two graphs. SARS-CoV-2 titers in supernatants collected from infected Vero E6 cells (multiplicity of infection [MOI]=0.1) treated with the indicated CR-31-B (−) concentrations were collected at 24 hours (h) post infection (p.i.) (n=6), and virus titers were determined by plaque assay (top). Significance levels compared to the results for untreated cells were determined by the two-tailed Mann Whitney U-test, a nonparametric test of the null hypothesis, and are indicated as follows: *, P<0.05; **, P<0.005. Data from six independent experiments were used to calculate the EC50 (EC₅₀) value by non-linear regression analysis (bottom). FIG. 18C depicts representative Western blot analysis of SARS-CoV-2 N protein accumulation (top panel) after treatment with the two enantiomers CR-31-B (−) and CR-31-B (+). Vero E6 cells were infected with SARS-CoV-2 (MOI=1) or left uninfected and treated with the indicated CR-31-B concentrations for 24 h p.i. Protein accumulation was analyzed by Western blotting using polyclonal rabbit anti-SARS nucleocapsid protein antibody (ROCKLAND™) and mouse anti-actin antibody (ABCAM™), respectively, each diluted 1:500 in PBS containing 1% bovine serum albumin (BSA). Beta (β)-actin (lower panel) was used as a loading control (n=3). FIG. 18D depicts representative immunofluorescence analysis to determine the effects of CR-31-B (+) and CR-31-B (−) on viral double-stranded RNA (dsRNA) accumulation in SARS-CoV-2-infected Vero E6 cells. Cells were infected (MOI=1) and cultivated in medium containing the indicated CR-31-B concentrations. Cells were fixed at 24 h p.i. and analyzed by confocal microscopy using a mouse anti-dsRNA mAb (J2, SCICONS ENGLISH & SCIENTIFIC CONSULTING KFT.™, red) that detects a viral RNA replication intermediate (red). Cell nuclei were stained with 4′,6-diamidino-2-phenylindole (DAPI; 2-(4-amidinophenyl)-1H-indole-6-carboxamidine) (blue) (n=2).

FIGS. 19A-19D are a schematic and graphs depicting a comparison of antiviral effects of CR-31-B (−) and Silvestrol using differentiated normal human bronchial epithelial (NHBE) cells infected with SARS-CoV-2. FIG. 19A is a schematic depicting how NHBE cells were cultivated and differentiated into different cell types (Clara, ciliated, goblet, and basal cells) under air-liquid interface conditions. NHBE cells (LONZA™, CC-2540) obtained from a 13-year-old Caucasian boy (Donor 1) and a 36-year-old Caucasian man (Donor 2), who were both non-smoking and lacking respiratory pathology, were seeded on collagen IV-coated transwell plates (CORNING COSTAR™, CLS3470-48EA) and grown in a mixture of DMEM (Invitrogen) and bronchial epithelial cell growth medium (BEGM) (LONZA™; CC-4175) supplemented with retinoic acid (75 nM). After reaching confluence, the cells were cultivated under air-liquid conditions for at least four additional weeks to allow differentiation into pseudostratified human airway epithelia (FIGS. 19B, 19C). Differentiated NHBE cells were infected with SARS-CoV-2 (MOI=3) and untreated (FIG. 19B), treated with Silvestrol (FIG. 19B), or treated with either CR-31-B (−) or CR-31-B (+) (FIGS. 19C-19D) at the indicated concentrations. At the indicated time points post infection (p.i), the apical surface of the cells was incubated with 100 microliters (100 L) PBS for 15 min, and SARS-CoV-2 titers in the cell culture supernatants were determined by virus plaque assay.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Disclosed herein are methods of treating a viral infection in a host cell or organism infected by the virus, the methods comprising administering to the cell or organism a therapeutically effective amount of a pharmaceutical composition comprising CR-31-B or a pharmaceutically acceptable salt thereof.

In some embodiments, the CR-31-B comprises a racemic mixture of:
i. a CR-31-B (−) enantiomer having the formula

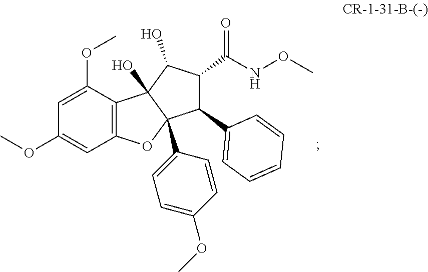

and
ii. a CR-31-B (+) enantiomer having the formula

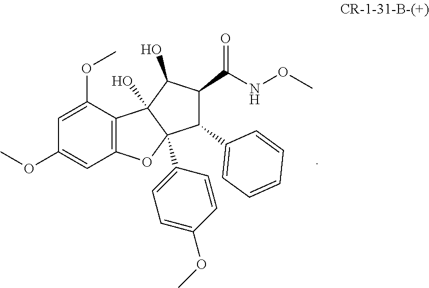

In other embodiments, the CR-31-B comprises at least 50% CR-31-B (−) enantiomer. In other embodiments, the CR-31-B is a CR-31-B (−) enantiomer.

In some embodiments, the CR-31-B reduces or inhibits a eukaryotic initiation factor 4A (eIF4A) activity. In some embodiments, the CR-31-B reduces or inhibits an eIF4A helicase activity. In some embodiments, the CR-31-B reduces or inhibits eIF4A clamping to a 5′-untranslated region (5′-UTR) of the mRNA of the virus. In some embodiments, the 5′-UTR comprises a hairpin structure. In some embodiments, the 5′-UTR comprises a polypurine sequence element comprising at least 5 purine nucleotides. In some embodiments, the polypurine sequence element comprises at least 20 purine nucleotides. In some embodiments, the polypurine sequence element comprises at least 30 purine nucleotides.

In some embodiments, the virus is an RNA virus.

In some embodiments, the virus comprises a virus from the Coronaviridae family, the Arenaviridae family, the Nairoviridae family, the Flaviviridae family, the Hepeviridae family, the Filoviridae family, or the Togaviridae family.

In some embodiments, the virus from the Coronaviridae family comprises human coronavirus 229E (HCoV-229E) (human common cold coronavirus), Middle East respiratory syndrome coronavirus (MERS-CoV), severe acute respiratory syndrome coronavirus (SARS-CoV), severe acute respiratory syndrome coronavirus 2 (SARS-CoV-2, COVID-19 virus), human coronavirus OC43 (HCoV-OC43), human coronavirus NL63 (HCoV-NL63), or human coronavirus HKU1 (HCoV-HKU1). Since 2000, there have been three documented cases of a coronavirus outbreak of zoonotic origin to reach epidemic or pandemic scale. These outbreaks have been caused by three severe Coronaviridae viruses: severe acute respiratory syndrome coronavirus (SARS-CoV or SARS-CoV-1) in 2002-2003; Middle East respiratory syndrome (MERS)-related coronavirus (MERS-CoV) in 2012-2013 (which is still ongoing); and severe acute respiratory syndrome coronavirus (SARS-CoV-2), a positive-sense, single-stranded RNA coronavirus of the genus Betacoronavirus in the subgenus Sarbecovirus, which is of zoonotic origin, causes a potentially severe respiratory disease with varying symptoms referred to as coronavirus disease 2019 (COVID-19) and is responsible for a pandemic starting in early 2020.

In some embodiments, the virus from the Arenaviridae family comprises Lassa mammarenavirus (LASV), Guanarito mammarenavirus, Junin mammarenavirus, Lujo mammarenavirus, Machupo mammarenavirus, Sabia mammarenavirus, or Whitewater Arroyo mammarenavirus.

In some embodiments, the virus from the Nairoviridae family comprises Crimean-Congo hemorrhagic fever virus (CCHFV).

In some embodiments, the virus from the Flaviviridae family comprises Zika virus (ZIKV), hepacivirus C (hepatitis C virus, HepC), dengue fever virus, yellow fever virus, Japanese encephalitis virus, or West Nile virus.

In some embodiments, the virus from the Hepeviridae family comprises hepatitis E virus (HEV) or hepatitis B virus.

In some embodiments, the virus from the Filoviridae family comprises Ebolavirus, Marburgvirus, Dianlovirus, Cuevavirus, Striavirus, or Thamnovirus.

In some embodiments, the virus from the Togaviridae family comprises an Alphavirus. In some embodiments, the virus from the Alphavirus comprises Chikungunya virus, Eastern equine encephalitis virus, Western equine encephalitis virus, Barmah Forest virus, Mayaro virus, O'nyong'nyong virus, Ross river virus, Semliki Forest virus, Sindbis virus, Una virus, Tonate virus, or Venezuelan equine encephalitis.

In some embodiments, the CR-31-B may be administered prophylactically before infection, may be administered after suspected or known virus exposure but prior to the appearance of symptoms of infection, administered during an incubation period of a virus, or any combination thereof.

In some embodiments, the composition further comprises a pharmaceutically acceptable carrier, excipient, or diluent.

Also disclosed herein are methods for reducing or inhibiting translation initiation of a messenger ribonucleic acid (mRNA) of a virus in a host cell or organism infected by the virus, the methods comprising administering to the cell or organism a therapeutically effective amount of a pharmaceutical composition comprising CR-31-B or a pharmaceutically acceptable salt thereof.

In some embodiment, the virus is an RNA virus.

Also disclosed herein are uses of a synthetic rocaglate composition for reducing or inhibiting translation initiation of a messenger ribonucleic acid (mRNA) of a virus in a host cell or organism infected by the virus, the synthetic rocaglate composition comprising a therapeutically effective amount of CR-31-B or a pharmaceutically acceptable salt thereof.

In some embodiments, the virus is an RNA virus.

Gene expression in prokaryotic and eukaryotic cells includes the steps of transcription of deoxyribonucleic acid (DNA) into ribonucleic acid (RNA). Transcription and subsequent processing of messenger RNA (mRNA) results in a template for protein synthesis via translation of the mRNA into protein, which is then further processed. In eukaryotes protein synthesis includes initiation, elongation, and termination steps. Part of the initiation phase includes the binding and subsequent activity of initiation factors.

A eukaryotic example of an initiation factor, the DEAD-box RNA helicase eukaryotic initiation factor 4A (eIF4A), which is part of the heterotrimeric translation initiation complex eukaryotic initiation factor 4F (eIF4F), unwinds ribonucleic acid (RNA) secondary structures in 5′-untranslated repeats (5′-UTRs) of selected messenger ribonucleic acids (mRNAs) to enable binding of the 43S preinitiation complex (PIC). In cells, eIF4A plays a role in the translation of protooncogenic messenger ribonucleic acids (mRNAs) with complex-structured 5′-UTRs.

Many viral RNAs contain 5′-UTRs with stable RNA structures (Madhugiri et al.

Adv. Virus Res. 96: 127-163; Schlereth et al. [2016] RNA Biol. 13: 783-798) and are thus dependent on eIF4A for translation. Viral protein synthesis is a host function critical to viral proliferation, and inhibition of viral protein synthesis can inhibit viral proliferation in the host.

Hallmark features of eIF4A-dependent translation define specific 5′-UTR elements that confer a requirement for the eIF4A RNA helicase. The key features are longer 5′-UTRs, a 12-mer (GGC)4 motif, and related 9-mer variant motifs. Importantly, the 12-mer and 9-mer motifs precisely localize to between 53% and 65% of all predicted RNA G-quadruplex structures (depending on the analysis tool). The 9-mer sequences require neighboring nucleotides to complete the structure as the minimal number is 12 nucleotides, and it was frequently observed that more than 12 nucleotides contribute to the G-quadruplex. Moreover, most of the remaining G-quadruplexes are based on highly similar sequence elements.

In contrast, internal ribosome entry site (IRES) mRNAs are somewhat protected, while cis-regulatory elements, such as 5′-terminal oligopyrimidine (TOP), 5-terminal oligopyrimidine-like (TOP-like), or pyrimidine-rich translational element (PRTE), do not appear to influence the eIF4A requirement. This is distinct from mammalian target of rapamycin complex 1 (mechanistic target of rapamycin complex 1; mTORC1) inhibition, which affects a different set of transcripts marked by TOP and TOP-like elements. For example, IRESs are 5′-UTR structural elements comprising stem-loop and pseudoknot structures that allow for an alternative method of cap- and 5′-end-independent translation initiation. However, TOP mRNAs contain a 5′-terminal oligopyrimidine tract (5′-TOP), encode for ribosomal proteins and eukaryotic elongation factors 1-alpha and 2 (eEF-1A and eEF-2), and are candidates for growth-dependent translational control mediated through their 5′-TOP, a sequence of 6-12 pyrimidines at the 5′-end. The mTOR Complex 1 (mTORC1) is a protein complex composed of mTOR itself, regulatory-associated protein of mTOR (commonly known as raptor), mammalian lethal with SEC13 protein 8 (MLST8), PRAS40 and DEPTOR. This complex embodies the classic functions of mTOR, namely as a nutrient/energy/redox sensor and controller of protein synthesis. These findings identify sequence motifs that represent translational control elements encoded in the 5′-UTR of several hundred transcripts and that confer a requirement for eIF4A RNA helicase action.

RNA G-quadruplex structures are typically made from at least two stacks of four guanosines exhibiting non-Watson-Crick interactions (e.g., hydrogen bonds) and connected by one or more linker nucleotides. The linker is most often a cytosine and less frequently an adenosine. There is variation in the exact structural composition and sequence requirement as our examples illustrate. The minimum requirement for the structure is a (GGC/A)4 sequence and neighboring nucleotides can complete the structure.

The cap-binding protein eIF4E is limiting for cap-dependent translation and its signaling is controlled by, e.g., mTORC1 and eukaryotic translation initiation factor 4E-binding protein 1 (4E-BP). mTORC1 activates transcription and translation through its interactions with p70-S6 Kinase 1 (S6K1) and 4E-BP1, the eukaryotic initiation factor 4E (eIF4E) binding protein 1. Their signaling converges at the translation initiation complex on the 5′ end of mRNA, and thus activates translation. Activated mTORC1 will phosphorylate translation inhibitor 4E-BP1, releasing it from eukaryotic translation initiation factor 4E (eIF4E). eIF4E is now free to join the eukaryotic translation initiation factor 4G (eIF4G) and the eukaryotic translation initiation factor 4A (eIF4A). This complex then binds to the 5′ cap of mRNA and recruits the helicase eukaryotic translation initiation factor A (eIF4A) and its cofactor eukaryotic translation initiation factor 4B (eIF4B). The helicase is required to remove hairpin loops that arise in the 5′ untranslated regions of mRNA, which prevent premature translation of proteins. Once the initiation complex is assembled at the 5′ cap of mRNA, it recruits the 40S small ribosomal subunit that is now capable of scanning for the AUG start codon start site, because the hairpin loop has been eradicated by the eIF4A helicase. Once the ribosome reaches the AUG codon, translation can begin. Hypophosphorylated S6K is located on the eIF3 scaffold complex. Active mTORC1 is recruited to the scaffold, and once there, phosphorylates S6K activate it mTORC1 phosphorylates S6K1 on at least two residues, with the most critical modification occurring on a threonine residue (T389). This event stimulates the subsequent phosphorylation of S6K1 by PDPK1. Active S6K1 can in turn stimulate the initiation of protein synthesis through activation of S6 Ribosomal protein (a component of the ribosome) and eIF4B, causing them to be recruited to the pre-initiation complex. For a set of mRNAs, the eIF4A helicase activity is required and represents the point of attack for three natural compounds, Silvestrol, hippuristanol, and pateamine. Regulatory interactions occur between eIF4A and the eIF4B, eIF4G, and eIF4H factors, and between S6 kinase in the phosphorylation and signaling control of eIF4B. These interactions define a broadly relevant layer of translational control that is distinct from the control of eIF4E by 4E-BP and mTORC1.

The two largest families of SF2 helicases, DEAD-box and DEAH-box proteins, share evolutionarily conserved helicase cores but unwind RNA helices through distinct mechanisms. A mechanism of translational control has been identified that is characterized by a requirement for eIF4A/DDX2 RNA helicase activity and underlies the antiviral effects of Silvestrol. The eukaryotic initiation factor-4A (eIF4A) family consists of 3 closely related proteins eIF4A1, eIF4A2, and eIF4A3. These factors are required for the binding of mRNA to 40S ribosomal subunits. In addition, these proteins are helicases that function to unwind double-stranded RNA. RNA helicases are essential for most processes of RNA metabolism such as ribosome biogenesis, pre-mRNA splicing, and translation initiation. They also play an important role in sensing viral RNAs. RNA helicases are involved in the mediation of antiviral immune response because they can identify foreign RNAs in vertebrates. About 80% of all viruses are RNA viruses and they contain their own RNA helicases. Defective RNA helicases have been linked to cancers, infectious diseases, and neuro-degenerative disorders. DEAD-box proteins, named for the amino acid sequence of a highly conserved motif, which include, but are not limited to, eIF4A1, eIF4A2, and eIF4A3, function primarily as ATP-driven, non-processive helicases, binding and unwinding short, exposed RNA duplexes before releasing the RNA and repeating the process on another duplex segment. In contrast, DEAH-box proteins share many sequence and structural similarities with DEAD-box proteins, but have a different mechanism of duplex unwinding. While DEAD-box proteins use simple cycles of RNA duplex binding and are highly specific for dsRNA, unwinding, and release, DEAH-box proteins function as translocating helicases, advancing in the 3′→5′ direction to disrupt nucleic acid structures, and some members of the DEAH-box family can act on both DNA and RNA, leading to unwinding of helices and, for some DEAH-box proteins, four-stranded G-quadruplex structures. Instead of binding directly to structured RNA elements, DEAH-box helicases require 3′ single-stranded regions for unwinding activity. DEAH-box proteins also lack specificity for ATP, binding and hydrolyzing all four NTPs to power cycles of directional movement. DEAH box proteins 9 and 36 (DHX9 and DHX36) are cytosolic helicases. DEAH-box protein helicases include, but are not limited to, DEAH box protein 9 (DHX9) and DEAH box protein 36 (DHX36). RNA helicases include, but are not limited to, eIF4A1, eIF4A2, eIF4A3, DHX9 or DHX36.

eIF4A-dependent translation-controlling motifs are typically present in the 5′-UTR of the mRNA. In certain embodiments, the eIF4A-dependent translation-controlling motif comprises a G-quadruplex structure.

In one embodiment, Silvestrol or CR-31-B interferes with eIF4A activity. In one embodiment, Silvestrol or CR-31-B inhibits eIF4A helicase activity.

“Rocaglates” are a class of compounds that act as potent inhibitors of translation initiation. In some embodiments, they are proposed to form stacking interactions with polypurine sequences in the 5′-untranslated region (UTR) of selected mRNAs, thereby clamping the RNA substrate onto eIF4A and causing inhibition of the translation initiation complex. Rocaglates include, but are not limited to, Silvestrol (methyl(1R,2R,3S,3aR,8bS)-6-[[(2S,3R,6R)-6-[(1R)-1,2-dihydroxyethyl]-3-methoxy-1,4-dioxan-2-yl]oxy]-1,8b-dihydroxy-8-methoxy-3a-(4-methoxyphenyl)-3-phenyl-2,3-dihydro-1H-cyclopenta[b][1]benzofuran-2-carboxylate), (±)-CR-31-B, among other rocaglamide ((1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide) derivatives. Silvestrol and at least some other natural rocaglates are derived from plants of the genus Aglaia in the mahogany family (Meliaceae).

Other compounds of interest include, but are not limited to, macrolides (e.g., pateamine A ((3S,6Z,8E,11S,15R,17S)-15-amino-3-[(1E,3E,5E)-7-(dimethylamino)-2, 5-dimethylhepta-1,3,5-trienyl]-9,11,17-trimethyl-4, 12-dioxa-20-thia-21-azabicyclo[16.2.1]henicosa-1(21),6,8,18-tetraene-5, 13-dione)) and steroids (e.g., hippuristanol).

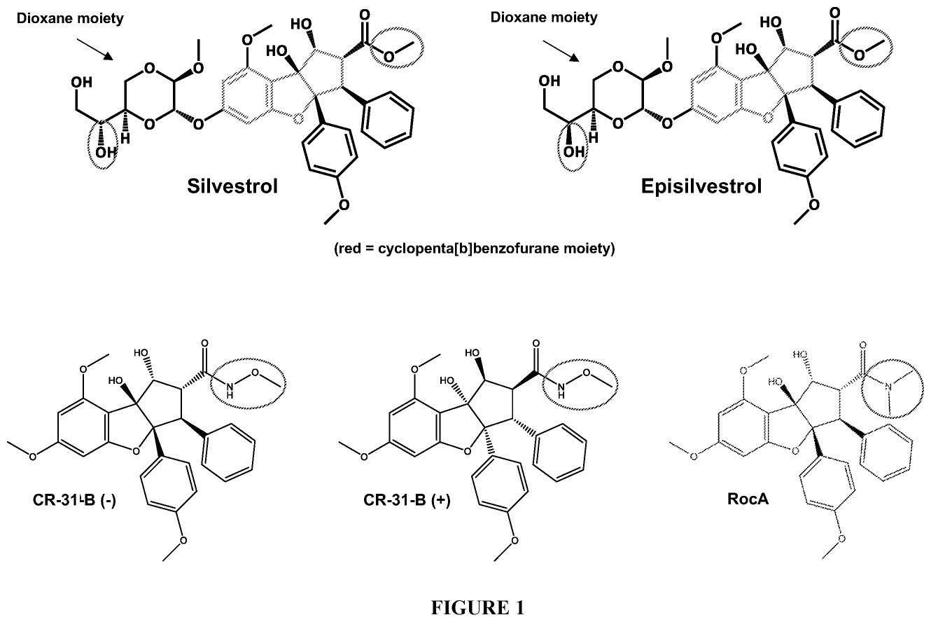

Synthetic rocaglates include, but are not limited to, CR-31-B (see FIG. 1). In some embodiments, the CR-31-B comprises a racemic mixture of:
i. a CR-31-B (−) enantiomer having the formula

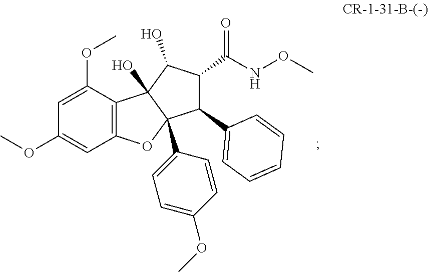

and
ii. a CR-31-B (+) enantiomer having the formula

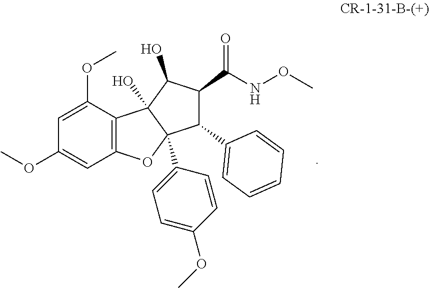

In other embodiments, the CR-31-B comprises at least 50% CR-31-B (−) enantiomer. In other embodiments, the CR-31-B is a CR-31-B (−) enantiomer.

Rocaglates (e.g., Silvestrol, CR-31-B) inhibit translation by reducing or inhibiting eIF4A activity. Reducing or inhibiting eIF4A activity can be achieved by reducing or inhibiting an eIF4A helicase activity and/or by reducing or inhibiting eIF4A clamping to a 5′-untranslated region (5′-UTR) of the mRNA of the virus. In some embodiments, the 5′-UTR comprises a hairpin structure. In some embodiments, the 5′-UTR comprises a polypurine sequence element comprising at least 20 purine nucleotides. In some embodiments, the polypurine sequence element comprises at least 30 purine nucleotides.

A “virus” is a small infectious agent. While not inside an infected cell or in the process of infecting a cell, viruses exist in the form of independent particles, or virions, consisting of: (i) the genetic material (i.e., long molecules of DNA or RNA that encode the structure of the proteins by which the virus acts; non-coding regions e.g. 5′-UTRs, 3′-UTRs or intergenic regions have regulatory functions during the life cycle of a virus); (ii) a protein coat, the capsid, which surrounds and protects the genetic material; and in some cases (iii) an outside envelope of lipids. A virus has either a DNA or an RNA genome and is called a “DNA virus” or an “RNA virus,” respectively. The majority of viruses have RNA genomes. Different viruses can infect prokaryotes or eukaryotes. An “RNA virus” usually has single-stranded RNA (ssRNA) as its genetic material, but may instead have double-stranded RNA (dsRNA) as its genetic material. RNA viruses can have a (+)-strand RNA genome or a (−)-strand RNA genome. (−)-RNA has to be transcribed into a (+)-strand RNA that contains the information for the synthesis of virus proteins. RNA viruses often have high mutation rates compared to DNA viruses, because viral RNA polymerases generally lack the proofreading ability of DNA polymerases. This high mutation rate often makes it difficult to construct effective vaccines against the diseases caused by RNA viruses.

Viruses cannot replicate on their own, but instead reproduce by infecting host cells and usurping the host cellular machinery, including the host transcription and/or translation machinery, to produce more virus particles. This property of viruses, as well as the ability of many viruses to mutate, makes treatment of viral infections difficult. Viral RNAs often contain highly structured 5′-UTRs, which may be eIF4A-dependent.

Viruses include, but are not limited to, coronaviruses, arenaviruses, bunyaviruses, flaviviruses, and orthohepeviruses. Viruses include, but are not limited to, viruses from the Coronaviridae family, the Arenaviridae family, the Nairoviridae family, the Flaviviridae family, the Hepeviridae family, the Filoviridae family, or the Togaviridae family. Viruses include, but are not limited to, RNA viruses for which viral protein synthesis is eIF4A-dependent.

In some embodiments, the virus is from the Bunyavirales order, including, but not limited to the Arenaviridae family and/or the Nairoviridae family.

In some embodiments, the virus from the Coronaviridae family comprises human coronavirus 229E (HCoV-229E) (human common cold coronavirus), Middle East respiratory syndrome coronavirus (MERS-CoV), severe acute respiratory syndrome coronavirus (SARS-CoV), severe acute respiratory syndrome coronavirus 2 (SARS-CoV-2, COVID-19 virus), human coronavirus OC43 (HCoV-OC43), human coronavirus NL63 (HCoV-NL63), or human coronavirus HKU1 (HCoV-HKU1).

In some embodiments, the virus from the Arenaviridae family comprises a mammarenavirus, including, but not limited to, Lassa mammarenavirus (LASV), Guanarito mammarenavirus, Junin mammarenavirus, Lujo mammarenavirus, Machupo mammarenavirus, Sabia mammarenavirus, and/or Whitewater Arroyo mammarenavirus.

In some embodiments, the virus from the Nairoviridae family comprises Crimean-Congo hemorrhagic fever virus (CCHFV).

In some embodiments, the virus is from the Flaviviridae family, including, but not limited to, the Flavivirus genus (e.g., Zika virus (ZIKV), dengue fever virus, yellow fever virus, Japanese encephalitis virus, or West Nile virus) and/or the Hepacivirus genus (e.g., hepacivirus C). In some embodiments, the virus from the Flaviviridae family comprises Zika virus (ZIKV), hepacivirus C (hepatitis C virus, HepC), dengue fever virus, yellow fever virus, Japanese encephalitis virus, or West Nile virus.

In some embodiments, the virus is from the Hepeviridae family, including, but not limited to, the Orthohepevirus genus. In some embodiments, the virus from the Hepeviridae family comprises hepatitis E virus (HEV) or hepatitis B virus.

In some embodiments, the virus is from the Filoviridae family, including, but not limited to the Ebolavirus genus (Ebola virus disease; e.g., Zaire ebolavirus, Bombali ebolavirus, Bundabugyo ebolavirus, Reston ebolavirus, Sudan ebolavirus, and Tai Forest ebolavirus), the Marburgvirus genus (Marburg virus disease; e.g., Marburg marburgvirus [Marburg virus (MARV), Ravn virus (RAVV)]), the Dianlovirus genus (Mengla virus disease; e.g., Mengla virus), the Cuevavirus genus (Lloviu virus disease; e.g., Lloviu cuevavirus), the Striavirus genus, and/or the Thamnovirus genus.

In some embodiments, the virus is from the Togaviridae family, including, but not limited to the Alphavirus genus (e.g., Chikungunya virus [Chikungunya virus disease], Eastern equine encephalitis virus [Eastern equine encephalitis], Western equine encephalitis virus [Western equine encephalitis], Barmah Forest virus, Mayaro virus, O'nyong'nyong virus, Ross River virus, Semliki Forest virus, Sindbis virus, Una virus, Tonate virus, Venezuelan equine encephalitis virus [Venezuelan equine encephalitis], and others).

The term “polynucleotide” as used herein encompasses single-stranded or double-stranded nucleic acid polymers. In certain embodiments, the nucleotides comprising the polynucleotide can be ribonucleotides or deoxyribonucleotides or a modified form of either general category of nucleotide (e.g., DNA or RNA).

The term “operably linked” encompasses components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein encompasses polynucleotide sequences that can affect expression or processing of coding sequences to which they are ligated or operably linked.

Provided herein are pharmaceutical compositions comprising a therapeutically effective amount of CR-31-B. In some embodiments, the pharmaceutical composition further comprises one or more pharmaceutically acceptable carriers, excipients and/or diluents.

“Pharmaceutically acceptable carriers” include any excipient which is nontoxic to the cell or subject being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions of the invention may be formulated in a variety of ways, including for example, solid, semi-solid (e.g., cream, ointment, and gel), and liquid dosage forms, such as liquid solutions (e.g., topical lotion or spray), dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Pharmaceutical compositions suitable for use include sterile aqueous solutions (where water soluble) or dispersions and sterile powders for the extemporaneous preparation of sterile solutions or dispersions. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of injectable compositions can be brought about by including in the composition an agent which delays absorption, for example, aluminum monostearate and gelatin.

Sterile solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art.

Further, the preparations may be packaged and sold in the form of a kit. Such articles of manufacture will preferably have labels or package inserts indicating that the associated compositions are useful for treating a subject suffering a viral infection as described herein.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human, but non-human organisms, including non-human mammals and birds, as well as transgenic organisms, can also be treated. Treatment dosages may be titrated using routine methods known to those of skill in the art to optimize safety and efficacy.

In some embodiments, the compositions of the present invention may be administered prophylactically before infection, may be administered after suspected or known virus exposure but prior to the appearance of symptoms of infection, administered during an incubation period of a virus, or any combination thereof.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, species, age, sex, and weight of the individual, and the ability of the molecule to elicit a desired response in the individual. A therapeutically effective amount is also one in which any toxic or detrimental effects of the molecule are outweighed by the therapeutically beneficial effects.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples (untreated with the composition) can be assigned a relative activity value of 100%. A change in activity caused by the composition can be measured in the assays.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

As used herein, a compound “inhibits” an activity if the compound reduces the desired activity by at least 10% relative to the activity under the same conditions but lacking only the presence of the compound. The activity may be measured by any reproducible means. The activity may be measured in vitro or in vivo. In some embodiments, compounds used in the methods described herein inhibit a eIF4A activity by at least about 20%, by at least about 25%, by at least about 30%, by at least about 40%, by at least about 50%, by at least about 60%, by at least about 70%, by at least about 75%, by at least about 80%, by at least about 90%, by about 95%, by about 98%, or by about 99% or more.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, topical, transdermal, oral (for example, in capsules, suspensions or tablets), parenteral (including subcutaneous, intravenous, intramedullary, intraarticular, intramuscular, or intraperitoneal injection), or rectal. Administration to a subject may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In some embodiments, the subject is male human or a female human.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

Compounds of the invention can also be prepared as prodrugs, for example pharmaceutically acceptable prodrugs. The terms “pro-drug” and “prodrug” are used interchangeably herein and may refer to any compound which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form.

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

“Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to ±10%.

When not otherwise stated, “substantially” means “being largely, but not wholly, that which is specified” (e.g., “substantially pure”).

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Any patent, patent application publication, or scientific publication, cited herein, is incorporated by reference herein in its entirety.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Objective:

Comparison of broad-spectrum antiviral activities of the synthetic rocaglates CR-31-B (−), CR-31-B (+), and CR-31-B (+/−) with the eIF4A-inhibitor Silvestrol

### Materials & Methods

Cell Culture. Human lung fibroblasts (MRC-5), human lung carcinoma cells (A549) and murine hepatocytes (Hepa1-6 cells) were grown in Dulbecco's modified Eagle's medium (DMEM) supplemented with 10% fetal bovine serum (FBS), 100 U/ml penicillin, and 100 g/ml streptomycin at 37° C. and in an atmosphere containing 5% CO₂. Human dermal fibroblast cell lines, human dermal cancer cell lines, and human liver cancer cell lines were cultured as recommended by the American Type Culture Collection (ATCC®). Human dermal fibroblast (HDF) cells were cultured in Fibroblast Basal Medium (ATCC® PCS-201-030™) supplemented with Fibroblast Growth Kit—Low Serum (ATCC® PCS-201-041™). Human dermal cancer cell lines (COLO-829, HS294T, and SK-MEL-31) and human liver cancer cell lines (HuH-1, SK-HEP-1, SNU-475, PLC/PRF/5, HuH-7, SNU-182, and HepG2) were grown in Dulbecco's modified Eagle's medium (DMEM) supplemented with 10% fetal bovine serum (FBS), 100 U/ml penicillin, and 100 g/ml streptomycin. Vero E6 cells (African green monkey kidney epithelial cells) were grown in Dulbecco's modified Eagle's medium (DMEM) supplemented with 10% fetal bovine serum (FBS), 100 U/ml penicillin, and 100 micrograms/milliliter (g/ml) streptomycin at 37° C. in an atmosphere containing 5% CO₂. HepG2 cells were cultured in Iscove's Modified Dulbecco's Medium (IMDM) supplemented with 10% fetal calf serum (FCS) at 37° C. and 5% CO₂.

Reagents. Silvestrol was obtained from the Sarawak Biodiversity Centre (Kuching; North-Borneo, Malaysia; purity >99%). A 6 mM stock solution was prepared in DMSO (sterile-filtered; ROTH™) and diluted in DMEM or IMDM. Control cells were treated with corresponding DMSO dilutions lacking Silvestrol. CR-31-B (−) and/or CR-31-B (+) (Wolfe et al. [2014] Nature 513: 65-70) were dissolved in DMSO for a total concentration of 10 mM (individual enantiomer or total racemic mixture) and stored at −20° C.

Primers. Primers utilized in various examples are found in Table 1, Table 2, and Table 3.

TABLE 1
Primer sequence used for PCR-based site-directed mutagenesis of
pFR_HCV_xb constructs.

| Construct | Primer_fwd (5′-3′) | Primer_rev (5′-3′) |
|---|---|---|
| (AG)₁₅ | AGAGAGAGAGAGAGAGAGAGAG | TTAAGCGGGTCGCTGC |
| | AGAGAGAGATGGAAGACGCCAAA | AGG (SEQ ID NO: 2) |
| | AACATAAAGAAAGG (SEQ ID NO: | |
| | 1) | |
| (AC)₁₅ | ACACACACACACACACACACACA | TTAAGCGGGTCGCTGC |
| | CACACACATGGAAGACGCCAAAA | AGG (SEQ ID NO: 2) |
| | ACATAAAG (SEQ ID NO: 3) | |
| HEVgt3c | gcagaccacgtatgtggtcgatgccATGGAAGA | TTAAGCGGGTCGCTGC |
| | CGCCAAAAACATAAAGAAAGG | AGG (SEQ ID NO: 2) |
| | (SEQ ID NO: 4) | |
| HEVgt3c- | gcacaccacgtatgtggtcgatgccATGGAAGA | TTAAGCGGGTCGCTGC |
| G4C | CGCCAAAAACATAAAGAAAGG | AGG (SEQ ID NO: 2) |
| | (SEQ ID NO: 5) | |
| HEVgt3c- | gcaCaacacgtatgtggtcgatgccATGGAAGA | TTAAGCGGGTCGCTGC |
| G4CC6A | CGCCAAAAACATAAAGAAAGG | AGG (SEQ ID NO: 2) |
| | (SEQ ID NO: 6) | |
| HEVgt3c- | ggagaggacgtatgtggtcgatgccATGG (SEQ | TTAAGCGGGTCGCTGC |
| Purine | ID NO: 7) | AGG (SEQ ID NO: 2) |
| VP35-HP | ATGGAAGACGCCAAAAACATAAA | gatgaaggttttaatcttcatcTTAA |
| only | GAAAGG (SEQ ID NO: 8) | GCGGGTC (SEQ ID NO: 9) |
| VP35- | AGAGATGGAAGACGCCAAAAACA | TCTCTgatgaaggttttaatcttcat |
| HP + (AG)₅ | TAAAG (SEQ ID NO: 10) | CTTAAGCGG (SEQ ID |
| | | NO: 11) |

TABLE 2
Primer sequences used for PCR-based Gibson Assembly of
pFR_HCV_xb constructs.

| Construct | Primer_fwd (5′-3′) | Primer_rev (5′-3′) |
|---|---|---|
| HCoV- | Primer fragment_fwd (5′-3′) | Primer fragment_rev (5′-3′) |
| 229E | CCCTGCAGCGACCCGCTTAAAC | ATGTTTTTGGCGTCTTCC |
| | TTAAGTACCTTATCTATCTACAG | ATTAGGAACAGTTACTGG |
| | ATAGAAAAGTTGCT (SEQ ID NO: | TTTCCACAAAGAC (SEQ |
| | 12) | ID NO: 13) |
| | Primer vector_fwd (5′-3′) | Primer vector_rev (5′-3′) |
| | GAAACCAGTAACTGTTCCTAAT | GATAGATAAGGTACTTAA |
| | GGAAGACGCCAAAAACATAAA | GTTTAAGCGGGTCGCTGC |
| | GAAAG (SEQ ID NO: 14) | AGG (SEQ ID NO: 15) |
| MERS | Primer fragment_fwd (5′-3′) | Primer fragment_rev (5′-3′) |
| | CCCTGCAGCGACCCGCTTAAgatt | ATGTTTTTGGCGTCTTCC |
| | taagtgaatagcttggctatctcacttcc | ATgatgtgccccgaattgccac (SEQ |
| | (SEQ ID NO: 16) | ID NO: 17) |
| | Primer vector_fwd (5′-3′) | Primer vector_rev (5′-3′) |
| | gtggcaattcggggcacatcATGGAAGAC | ccaagctattcacttaaatcTTAAGC |
| | GCCAAAAACATAAAGAAAG | GGGTCGCTGCAGGGT |
| | (SEQ ID NO: 18) | (SEQ ID NO: 19) |
| VP30 | Primer fragment_fwd (5′-3′) | Primer fragment_rev (5′-3′) |
| | CCCTGCAGCGACCCGCTTAAgatg | ATGTTTTTGGCGTCTTCC |
| | aagattaagaaaaaggtaatctttcgattatcttt | ATttaaaagttgtctccactcactgtttaaa |
| | aatcttc (SEQ ID NO: 20) | tttct (SEQ ID NO: 21) |
| | Primer vector_fwd (5′-3′) | Primer vector_rev (5′-3′) |
| | tgagtggagacaacttttaaATGGAAGACG | cctttttcttaatcttcatcTTAAGCGG |
| | CCAAAAACATAAAGAAAG (SEQ | GTCGCTGCAGG (SEQ ID |
| | ID NO: 22) | NO: 23) |
| VP35 | Primer fragment_fwd (5′-3′) | Primer fragment_rev (5′-3′) |
| | CCCTGCAGCGACCCGCTTAAgatg | ATGTTTTTGGCGTCTTCC |
| | aagattaaaaccttcatcatccttacgt (SEQ ID | ATcttgttagaccagcttttcttttacattga |
| | NO: 24) | (SEQ ID NO: 25) |
| | Primer vector_fwd (5′-3′) | Primer vector_rev (5′-3′) |
| | agaaaagctggtctaacaagATGGAAGAC | tgaaggttttaatcttcatcTTAAGCG |
| | GCCAAAAACATAAAGAAAG | GGTCGCTGCAGG (SEQ ID |
| | (SEQ ID NO: 26) | NO: 27) |
| SARS- | Primer fragment fwd (5-3′) | Primer fragment rev (5′-3) |
| CoV-2 | CCCTGCAGCGACCCGCTTAAAT | ATGTTTTTGGCGTCTTCC |
| | TAAAGGTTTATACCTTCCCAGG | ATCTTACCTTTCGGTCAC |
| | TAACAAACC (SEQ ID NO: 36) | ACCC (SEQ ID NO: 37) |
| | Primer vector fwd (5′-3) | Primer vector rev (5′-3) |
| | GGGTGTGACCGAAAGGTAAGA | GGAAGGTATAAACCTTTA |
| | TGGAAGACGCCAAAAACATAA | ATTTAAGCGGGTCGCTGC |
| | AGAAAG (SEQ ID NO: 38) | AGGGTCGC (SEQ ID NO: 39) |

TABLE 3
Additional primers.

| Primer Type | | |
|---|---|---|
| Genomic | HCOV nsp1s (5′-3′) | HCOV nsp1as (5′-3′) |
| Primers | AAGCCGTCCGCCGTTATAG | CAAATCGGCATGCCCT |
| | (SEQ ID NO: 28) | AAA (SEQ ID NO: 29) |
| N-Primer | nuc_s (5′-3′) | nuc_as (5′-3′) |
| HCoV | cggtgttaggcgcaagaatt | aggagcacgggagtcaggtt |
| | (SEQ ID NO: 30) | (SEQ ID NO: 31) |
| GAPDH | GAP_s (5′-3′) | GAP_as (5′-3′) |
| Housekeeping | CTGCTCCTCCTGTTCGACAGT | CCGTTGACTCCGACCT |
| Primers | (SEQ ID NO: 32) | TCAC (SEQ ID NO: 33) |

Cell toxicity. Cell growth and viability of murine hepatocytes and MRC-5 cells in the presence of the respective compounds was determined by ATPLITE™ assay (PERKIN ELMER™) or the 3-(4,5-dimethylthiazol-2-yl)-2,5-diphenyl-2H-tetrazoliumbromide (MTT) method as described previously (Günther et al. [2004] Antivir. Res. 63: 209-215; Müller et al. [2018a] Antivir. Res. 150: 123-129). The cytotoxic concentration 50% (CC₅₀) of CR-31-B was determined as described (Müller et al. [2018a] Antivir. Res. 150: 123-129). A sigmoidal dose-response curve was fitted to the data using PRISM GRAPHPAD™ 6.0 (GRAPHPAD SOFTWARE™). The inhibitory concentrations that reduced the virus titer by 50%, (IC₅₀,) were calculated from the sigmoidal functions. Cell viability of A549 cells persistently infected with HEV was determined using the PRESTOBLUE™ Cell Viability Reagent (THERMOFISHER SCIENTIFIC™) after treatment with the substances in the respective concentrations for 72 h. To determine CC₅₀ values for human dermal fibroblast cells, human dermal cancer cell lines, and human liver cancer cell lines, cell lines were treated with racemic (+/−) CR-31-B for 48 h, and cell viability was measured by adenosine triphosphate (ATP) quantification using the CELLTITER-GLO™ Luminescent Cell Viability Assay (PROMEGA™ G7571). Cell viability of Vero E6 cells in the presence of the respective compounds was determined by MTT assay as described herein.

Human airway epithelial cells. Cryopreserved normal human bronchial epithelial (NHBE) cells were obtained from LONZA™. Undifferentiated cells were seeded on transwell plates (CORNING COSTAR™) coated with Collagen IV (INVITROGEN™) and grown in a mixture of DMEM (INVITROGEN™) and bronchial epithelial cell growth medium (BEGM) (LONZA™) supplemented with retinoic acid (75 nM). Every other day fresh medium was added. After reaching confluence, the cells were cultivated under air-liquid conditions for 4 additional weeks to differentiate into pseudostratified human airway epithelia. During this period, medium from the basolateral compartment was renewed every 2-3 days, and the apical surface was washed once per week with PBS (INVITROGEN™).

Viruses. High-titer stocks of HCoV-229E (NCBI accession number NC_002645) and MERS-CoV (EMC/2012; NCBI accession number NC_019843) were produced using Huh-7 cells. High-titer stocks of CCHFV strain Afg-09 2990 (Ölschlager et al. [2010]J. Clin. Virol. 50: 90-92) and LASV strain Ba366 (Lecompte et al. [2006] Emerg. Infect. Dis. 12: 1971-1974) were produced in Vero E6 cells. The ZIKV 976 Uganda (U) was kindly provided by the European Virus Archive. Persistently HEV-infected cells (gt3c strain 47832c; GenBank ID KC618403.1) were previously generated (Johne et al. [2014]J. Viral Hepat. 21:447-456).

Antiviral activity. To determine the antiviral activity of CR-31-B and Silvestrol, MRC5 cells or murine hepatocytes were inoculated with the respective virus at a multiplicity of infection (MOI) of 0.1 or 0.01 at 33° C. (HCoV-229E) or 37° C. (MERS-CoV, LASV, CCHFV). After 1 h, the inoculum was removed, and cells were incubated with fresh medium containing the inhibitor at increasing concentrations. Supernatants were collected at 24 h post infection (hpi; HCoV-229E, MERS-CoV) or 3 days post infection (dpi; LASV, CCHF), and virus titers were analyzed by virus plaque assay (Müller et al. [2018b]J. Virol. 92 pii: e01463-17) or immunofocus assay as described before (Gunther et al. [2004] Antivir. Res. 63: 209-215). To calculate effective concentration 50% (half-maximal effective concentration; EC₅₀) values, the virus titer determined for virus-infected cells treated with DMSO only was set to 100%, and titers obtained treated cells were calculated in relation to it. EC₅₀ values were calculated by non-linear regression analysis using GRAPHPAD PRISM™ 6.0 (GRAPHPAD SOFTWARE™).

For the infection of the primary human airway epithelial cells, the apical surface was washed 3 times with PBS before the cells were infected with HCoV-229E (MOI=3). After 1 h the inoculum was removed and the medium in the basal compartment was replaced with medium containing the indicated inhibitor concentrations. At the indicated time points, the apical surface of the cells was incubated with PBS for 15 min, and virus titers in the supernatants were determined by virus plaque assay. Effects on HEV were analyzed using persistently HEV-infected A549 cells. Treatments with the compounds were started 24 h post-seeding and cell culture supernatants were analyzed after 72 h via quantitative reverse transcription-polymerase chain reaction (qRT-PCR).

Infection of A549 cells with the ZIKV strain 976 Uganda (U) (provided by the European Virus Archive) was performed using a MOI of 0.1 for 16 h. Simultaneously, the cells were treated with compounds in the respective concentrations. Afterwards, the inoculum was removed, and cells were washed with PBS and treated with compounds for an additional 8 h. Cells were lysed in PEQGOLD TRIFAST™ (VWR™) 24 hpi to isolate intracellular RNA. After reverse transcription, the ZIKV genomes were quantified by qRT-PCR to determine the EC₅₀, as described above.

To determine the 50% effective concentration (EC₅₀) for CR-31-B (−), Vero E6 cells were inoculated with SARS-CoV-2 at a multiplicity of infection (MOI) of 0.1 at 33° C. After 1 h, the inoculum was removed, and cells were incubated with fresh medium containing CR-31-B (−) at increasing concentrations. Virus-containing supernatants were collected at 24 hours post infection (hpi) and virus titers were analyzed via plaque assay. EC50 values were determined based on virus titers in supernatants of infected cells treated with solvent control (DMSO) compared to virus titers in supernatants of infected cells treated with the respective inhibitor concentration. EC₅₀ values were then calculated by non-linear regression analysis using GRAPHPAD PRISM™ 6.0 (GRAPHPAD SOFTWARE™).

For the infection of normal human bronchial epithelial cells (NHBE) cells, the apical surface was washed 3 times with PBS and cells were infected with SARS-CoV-2 (MOI=3). After 1 h, the inoculum was removed and the medium in the basal compartment was replaced with medium containing the indicated inhibitor concentration. At the indicated time points post-infection (p.i.), the apical surface of the cells was incubated with PBS for 15 min and virus titers in the supernatants were determined by plaque assay.

Western blot analysis. To analyze viral protein accumulation, MRC-5 cells were infected with HCoV-229E at a MOI of 1. After inoculation, the supernatant was replaced with DMEM supplemented with antibiotics and the indicated concentrations of CR-31-B enantiomers. At 24 hpi, cell lysates were prepared, and viral proteins were analyzed by Western blotting (Müller et al. [2018a] Antivir. Res. 150: 123-129).

To analyze viral protein accumulation, Vero E6 cells were infected with SARS-CoV-2 at a multiplicity of infection (MOI) of 1. After inoculation, the supernatant was replaced with fresh medium supplemented with the indicated concentrations of the respective CR-31-B enantiomer. After 24 h, the medium was removed, the cells were washed with PBS and lysed using buffer containing 50 mM Tris-HCl (tris(hydroxymethyl)aminomethane-HCl), pH 7.5, 150 mM NaCl, 1% NP40 (nonyl phenoxypolyethoxylethanol), and 1× protease inhibitor cocktail (P8340; SIGMA-ALDRICH™). The insoluble material was removed by centrifugation and the protein content in the supernatant was measured using a QUBIT™ 3 fluorometer (INVITROGEN™) and equal amounts of proteins were separated in sodium dodecyl sulfate (SDS)-10% polyacrylamide gels and blotted onto a nitrocellulose membrane (AMERSHAM™). Membranes were incubated with polyclonal rabbit anti-SARS nucleocapsid protein antibody (ROCKLAND™) and mouse-anti actin antibody (ABCAM™), respectively, each diluted 1:500 in PBS containing 1% bovine serum albumin (BSA). After 60 min, membranes were washed with PBS and incubated with appropriate secondary antibodies (Fluorescent Dye (IRDye)-conjugated anti-mouse or anti-rabbit IgG mAb [LI-COR BIOSCIENCES™]) diluted 1:10.000 in PBS containing 1% BSA. After 1 hour (h), membranes were washed and analyzed using the LI-COR ODYSSEY™ imaging system.

qRT-PCR of HCoV-299E RNA, extracellular HEV RNA, or ZIKV RNA. MRC-5 cells were infected with a MOI of 1 and incubated for 24 h with the indicated inhibitor concentrations. Then, total cellular RNA was isolated using RNEASY™ kit (QIAGEN™), and quantitative RT-PCR was performed using 5 ng RNA and the LUNA UNIVERSAL PROBE ONE-STEP™ RT-qPCR Kit (NEW ENGLAND BIOLABS™ [NEB]). Sequences of primers used to amplify genomic and total viral RNA, respectively, and GAPDH mRNA are shown in Table 3. For the analysis of relative fold viral RNA expression in regard to inhibitor treatment, the delta-delta Ct method (using GAPDH as a reference) was used (Livak et al. [2001] Method. Methods 25: 402-408). Isolation and quantification of extracellular HEV RNA was performed as described previously (Glitscher et al. [2018] Viruses 10: E301).

Dual luciferase constructs. All constructs are based on the commercially available plasmid pFR_HCV_xb (ADDGENE™) and were produced using PCR-based site-directed mutagenesis. Primers were designed using SNAPGENE 4.1.9™ (GSL BIOTECH LLC™). Primer sequences are shown in Table 1 and Table 2. The respective 5′-UTRs were cloned downstream of the HSV-TK promotor directly followed by the firefly luciferase gene, an HCV IRES, and the Renilla luciferase gene. The total length of the analyzed 5′-UTRs, including single-stranded and double-stranded regions, ranges from 25 bp to 292 bp ((AG)₁₅/(AC)₁₅: 30 bp; poly(AC)_{12.5}-5′-(AG)_{2.5}/poly(AC)_{12.5}-mid(AG)_{2.5}: 30 bp; poly(AC)₁₀-5′-(AG)₅/poly(AC)₁₀-mid-(AG)₅: 30 bp; poly(AC)_{7.5}-5′-(AG)_{7.5}/poly(AC)_{7.5}-mid(AG)_{7.5}: 30 bp; poly(AC)₅-5′-(AG)₁₀/poly(AC)₅-mid-(AG)₁₀: 30 bp; EBOV VP30: 221 bp; EBOV VP35: 97 bp; EBOV VP35-HP only: 22 bp; EBOV VP35-HP+(AG)₅: 32 bp; HEVgt3c: 25 bp; HEVgt3c-G4C: 25 bp; HEVgt3c-G4CC6A: 25 bp; HEVgt3c-Purine: 25 bp; HCoV-229E: 292 bp; MERS-CoV: 278 bp; SARS-CoV-2. 265 bp).

Dual luciferase reporter assay. The dual luciferase reporter assay was performed as described previously (Müller et al. [2018a] Antivir. Res. 150: 123-129; Müller et al. [2020] Antivir. Res. 175: 104706). All experiments were performed in at least three independent replicates. The total length of the analyzed 5′-UTRs, including single-stranded and double-stranded regions, ranges from 25 bp to 292 bp ((AG)₁₅/(AC)₁₅: 30 bp; poly(AC)_{12.5}-5′-(AG)_{2.5}/poly(AC)_{12.5}-mid(AG)_{2.5}: 30 bp; poly(AC)₁₀-5′-(AG)₅/poly(AC)₁₀-mid-(AG)₅: 30 bp; poly(AC)_{7.5}-5′-(AG)_{7.5}/poly(AC)_{7.5}-mid(AG)_{7.5}: 30 bp; poly(AC)₅-5′-(AG)₁₀/poly(AC)₅-mid-(AG)₁₀: 30 bp; EBOV VP30: 221 bp; EBOV VP35: 97 bp; EBOV VP35-HP only: 22 bp; EBOV VP35-HP+(AG)₅: 32 bp; HEVgt3c: 25 bp; HEVgt3c-G4C: 25 bp; HEVgt3c-G4CC6A: 25 bp; HEVgt3c-Purine: 25 bp; HCoV-229E: 292 bp, MERS-CoV: 278 bp, SARS-CoV-2: 265 bp). Sequences for the 5′-UTRs of β-globin, HCoV-229E, MERS-CoV and SARS-CoV-2 are shown in Table 6.

TABLE 6
5′-Untranslated Regions (UTRs).

| 5′-UTR | Sequence (5′-3′) |
|---|---|
| (Nucleotide) | (SEQ ID NO:) |
| (AG)₁₅ | AGAGAGAGAGAGAGAGAGAGAGAGAGAGAG |
| | (SEQ ID NO: 48) |
| (AC)₁₅ | ACACACACACACACACACACACACACACAC (SEQ |
| | ID NO: 49) |
| Poly(AC)_{12.5}-5′-(AG)_{2.5} | AGAGACACACACACACACACACACACACAC (SEQ |
| | ID NO: 50) |
| Poly(AC)₁₀-5′-(AG)₅ | AGAGAGAGAGACACACACACACACACACAC (SEQ |
| | ID NO: 51) |
| Poly(AC)_{7.5}-5′-(AG)_{7.5} | AGAGAGAGAGAGAGACACACACACACACAC (SEQ |
| | ID NO: 52) |
| Poly(AC)₅-5′-(AG)₁₀ | AGAGAGAGAGAGAGAGAGAGACACACACAC (SEQ |
| | ID NO: 53) |
| PolyAC-mid-(AG)_{2.5} | ACACACACACACAGAGACACACACACACAC (SEQ |
| | ID NO: 54) |
| PolyAC-mid(AG)₅ | ACACACACACAGAGAGAGAGACACACACAC (SEQ |
| | ID NO: 55) |
| PolyAC-mid-(AG)_{7.5} | ACACACAGAGAGAGAGAGAGACACACACAC (SEQ |
| | ID NO: 56) |
| PolyAC-mid-(AG)₁₀ | ACACAGAGAGAGAGAGAGAGAGAGACACAC (SEQ |
| | ID NO: 57) |
| HEVgt3c | GCAGACCACGTATGTGGTCGATGCC (SEQ ID NO: |
| | 58) |
| HEVgt3c-G4C | GCACACCACGTATGTGGTCGATGCC (SEQ ID NO: |
| | 59) |
| HEVgt3c-G4CC6A | GCACAACACGTATGTGGTCGATGCC (SEQ ID NO: |
| | 60) |
| HEVgt3c-Purine | GGAGAGGACGTATGTGGTCGATGCC (SEQ ID NO: |
| | 61) |
| EBOV VP30 | GATGAAGATTAAGAAAAAGGTAATCTTTCGATTA |
| | TCTTTAATCTTCATCCTTGATTCTACAATCATGACA |
| | GTTGTCTTTAGTGACAAGGGAAAGAAGCCTTTTTA |
| | TTAAGTTGTAATAATCAGATCTGCGAACCGGTAGA |
| | GTTTAGTTGCAACCTAACACACATAAAGCATTGGT |
| | CAAAAAGTCAATAGAAATTTAAACAGTGAGTGGA |
| | GACAACTTTTAA (SEQ ID NO: 62) |
| EBOV VP35 | GATGAAGATTAAAACCTTCATCATCCTTACGTCAA |
| | TTGAATTCTCTAGCACTCGAAGCTTATTGTCTTCA |
| | ATGTAAAAGAAAAGCTGGTCTAACAAG (SEQ ID |
| | NO: 63) |
| VP35-HP only | GAUGAAGAUUAAAACCUUCAUC (SEQ ID NO: 64) |
| VP35-HP + (AG)₅ | GAUGAAGAUUAAAACCUUCAUCAGAGAGAGAG |
| | (SEQ ID NO: 65) |
| β-globin (beta-globin) | ACATTTGCTTCTGACACAACTGTGTTC |
| (DNA) | ACTAGCAACCTCAAACAGACACC |
| | (SEQ ID NO: 46) |
| β-globin (beta-globin) | ACAUUUGCUUCUGACACAACUGUGUUC |
| (RNA) | ACUAGCAACCUCAAACAGACACC |
| | (SEQ ID NO: 47) |
| HCoV-229E | ACTTAAGTACCTTATCTATCTACAGATA |
| (DNA) | GAAAAGTTGCTTTTTAGACTTTGTGTCT |
| | ACTTTTCTCAACTAAACGAAATTTTTGC |
| | TATGGCCGGCATCTTTGATGCTGGAGT |
| | CGTAGTGTAATTGAAATTTCATTTGGG |
| | TTGCAACAGTTTGGAAGCAAGTGCTGT |
| | GTGTCCTAGTCTAAGGGTTTCGTGTTC |
| | CGTCACGAGATTCCATTCTACAAACGC |
| | CTTACTCGAGGTTCCGTCTCGTGTTTG |
| | TGTGGAAGCAAAGTTCTGTCTTTGTGG |
| | AAACCAGTAACTGTTCCTA |
| | (SEQ ID NO: 40) |
| HCoV-229E | ACUUAAGUACCUUAUCUAUCUACAGAUA |
| (RNA) | GAAAAGUUGCUUUUUAGACUUUGUGUCU |
| | ACUUUUCUCAACUAAACGAAAUUUUUGC |
| | UAUGGCCGGCAUCUUUGAUGCUGGAGU |
| | CGUAGUGUAAUUGAAAUUUCAUUUGGG |
| | UUGCAACAGUUUGGAAGCAAGUGCUGU |
| | GUGUCCUAGUCUAAGGGUUUCGUGUUC |
| | CGUCACGAGAUUCCAUUCUACAAACGC |
| | CUUACUCGAGGUUCCGUCUCGUGUUUG |
| | UGUGGAAGCAAAGUUCUGUCUUUGUGG |
| | AAACCAGUAACUGUUCCUA |
| | (SEQ ID NO: 41) |
| MERS-CoV | GATTTAAGTGAATAGCTTGGCTATCTC |
| (DNA) | ACTTCCCCTCGTTCTCTTGCAGAACTTT |
| | GATTTTAACGAACTTAAATAAAAGCCC |
| | TGTTGTTTAGCGTATCGTTGCACTTGTC |
| | TGGTGGGATTGTGGCATTAATTTGCCT |
| | GCTCATCTAGGCAGTGGACATATGCTC |
| | AACACTGGGTATAATTCTAATTGAATA |
| | CTATTTTTCAGTTAGAGCGTCGTGTCTC |
| | TTGTACGTCTCGGTCACAATACACGGT |
| | TTCGTCCGGTGCGTGGCAATTCGGGGC |
| | ACATC |
| | (SEQ ID NO: 42) |
| MERS-CoV | GAUUUAAGUGAAUAGCUUGGCUAUCUC |
| (RNA) | ACUUCCCCUCGUUCUCUUGCAGAACUUU |
| | GAUUUUAACGAACUUAAAUAAAAGCCC |
| | UGUUGUUUAGCGUAUCGUUGCACUUGUC |
| | UGGUGGGAUUGUGGCAUUAAUUUGCCU |
| | GCUCAUCUAGGCAGUGGACAUAUGCUC |
| | AACACUGGGUAUAAUUCUAAUUGAAUA |
| | CUAUUUUUCAGUUAGAGCGUCGUGUCUC |
| | UUGUACGUCUCGGUCACAAUACACGGU |
| | UUCGUCCGGUGCGUGGCAAUUCGGGGC |
| | ACAUC |
| | (SEQ ID NO: 43) |
| SARS-CoV-2 | ATTAAAGGTTTATACCTTCCCAGGTAA |
| (DNA) | CAAACCAACCAACTTTCGATCTCTTGT |
| | AGATCTGTTCTCTAAACGAACTTTAAA |
| | ATCTGTGTGGCTGTCACTCGGCTGCAT |
| | GCTTAGTGCACTCACGCAGTATAATTA |
| | ATAACTAATTACTGTCGTTGACAGGAC |
| | ACGAGTAACTCGTCTATCTTCTGCAGG |
| | CTGCTTACGGTTTCGTCCGTGTTGCAG |
| | CCGATCATCAGCACATCTAGGTTTCGT |
| | CCGGGTGTGACCGAAAGGTAAG |
| | (SEQ ID NO: 44) |
| SARS-CoV-2 | AUUAAAGGUUUAUACCUUCCCAGGUAA |
| (RNA) | CAAACCAACCAACUUUCGAUCUCUUGU |
| | AGAUCUGUUCUCUAAACGAACUUUAAA |
| | AUCUGUGUGGCUGUCACUCGGCUGCAU |
| | GCUUAGUGCACUCACGCAGUAUAAUUA |
| | AUAACUAAUUACUGUCGUUGACAGGAC |
| | ACGAGUAACUCGUCUAUCUUCUGCAGG |
| | CUGCUUACGGUUUCGUCCGUGUUGCAG |
| | CCGAUCAUCAGCACAUCUAGGUUUCGU |
| | CCGGGUGUGACCGAAAGGUAAG |
| | (SEQ ID NO: 45) |

Immunofluorescence. Immunofluorescence was performed as described previously (Miller et al. [2018c]J. Virol. 92:(4): e01463-17. doi: 10.1128/JVI.01463-17). Briefly, Vero E6 cells were infected with SARS-CoV-2 (MOI of 1) and treated with the indicated concentrations of CR-31-B (−) or CR-31-B (+) for 24 hpi or left untreated. Then, the cells were fixed with ice-cold methanol and stained with mouse anti-dsRNA mAb (J2, SCICONS ENGLISH & SCIENTIFIC CONSULTING KFT.™). As secondary antibodies, ALEXAFLUOR™ 594 goat anti-mouse IgG was used. Confocal microscopy was done using a LEICA™ SP05 CLSM™ and LAS-AF™ software (LEICA™).

### Example 1: Antiviral Activity of CR-31-B (−) Against Coronavirus In Vitro

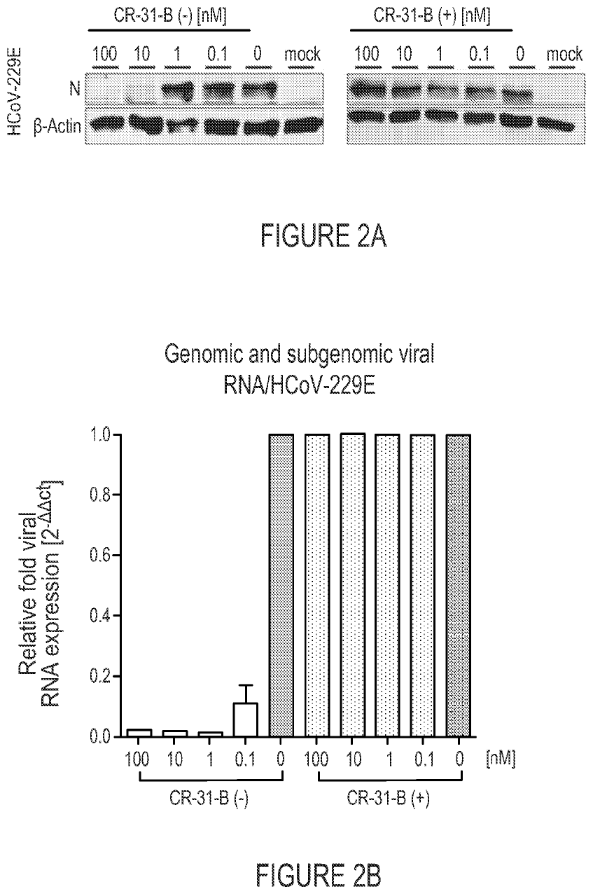

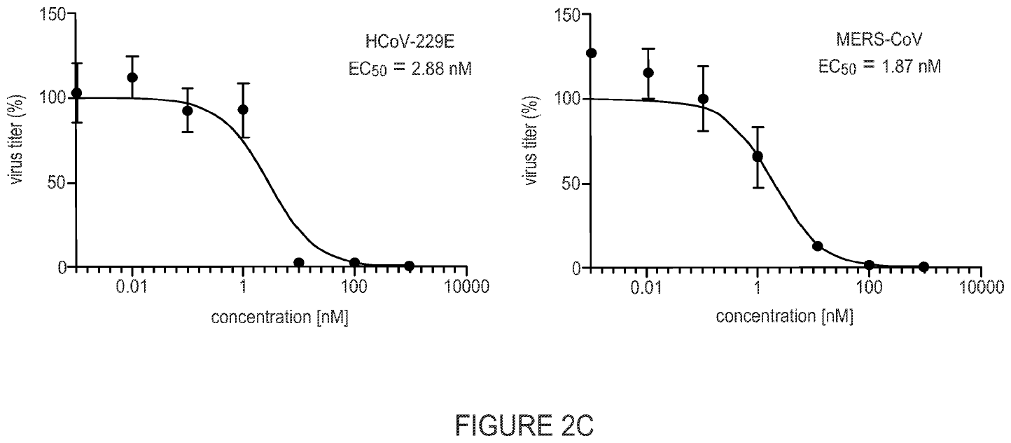

We have recently shown that Silvestrol can efficiently inhibit viral protein synthesis in MERS-CoV- and HCoV-229E-infected MRC-5 cells (Miller et al. [2018a] Antivir. Res. 150: 123-129). Here, we investigated the antiviral potential of CR-31-B, a synthetic rocaglate lacking the dioxane moiety of Silvestrol (FIG. 1). MRC-5 cells were infected with HCoV-229E at a MOI of 1 and the effects of the CR-31-B (−)- and (+)-enantiomers on viral protein synthesis were analyzed 24 hpi. Coronavirus N-protein levels were found to be strongly reduced in the presence of the (−)-enantiomer at concentrations >10 nM, while CR-31-B (+) had no detectable effect (FIG. 2A). Similarly, the genomic and subgenomic RNA levels of HCoV-229E were reduced in the presence of sub-nanomolar concentrations of CR-31-B (−) (FIG. 2B). In line with this, the formation of viral replication/transcription complexes was reduced in the presence of CR-31-B (−), as shown by immunofluorescence analysis using antibodies directed against nonstructural protein 8 (nsp8) and double-stranded RNA (dsRNA) (FIG. 3).

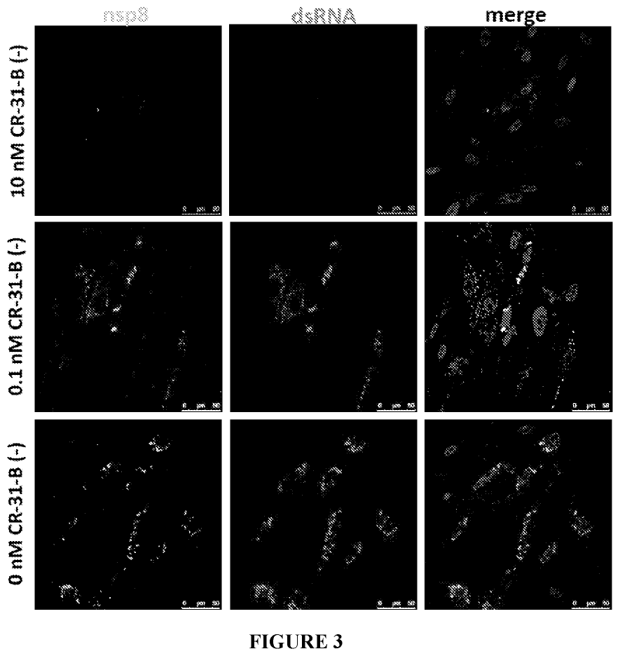

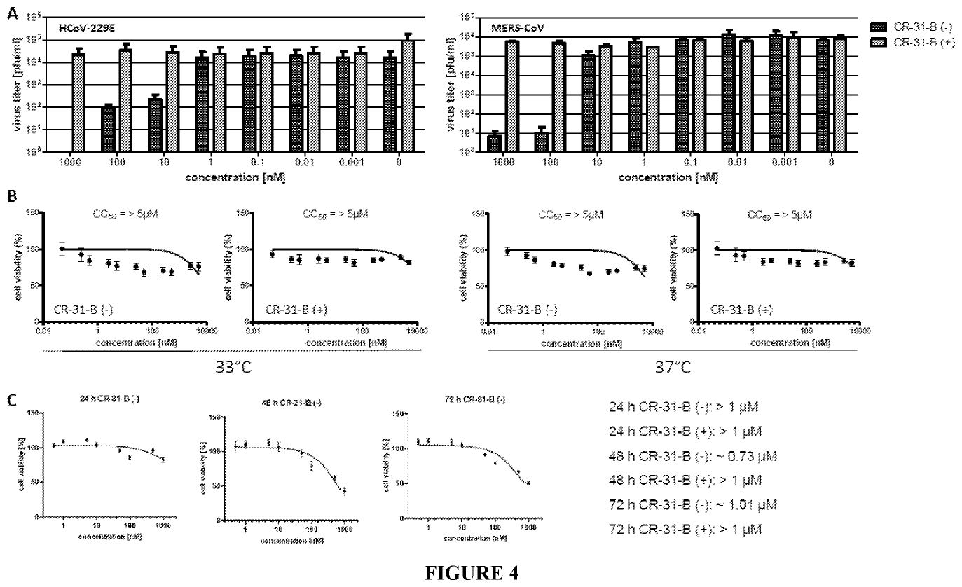

Next, we analyzed the effects of CR-31-B (−) on the production of coronavirus progency using HCoV-229E- and MERS-CoV-infected MRC-5 cells. For both viruses, CR-31-B (−) was revealed to reduce viral titers efficiently, with EC₅₀ values of 3.49 nM for HCoV-229E and 2.09 nM for MERS-CoV (FIG. 2C). At a concentration of 100 nM CR-31-B (−), the MERS-CoV titer was reduced about 5 log phases (FIG. 4A). Cytotoxicity tests using MRC-5 cells revealed that CR-31-B (−) and CR-31-B (+) caused a slight reduction of cell viability by 10-25% and 10%, respectively, if the cells were incubated for 24 h with concentrations of up to 5 μM of the respective compound (FIG. 4B), indicating low cytotoxicities for both compounds with selectivity indices of >1000 (see Table 4).

TABLE 4
CC₅₀ and EC₅₀ values determined for Silvestrol and CR-31-B
(−)-treated cells that were mock infected (CC₅₀) or infected
with the indicated viruses (EC₅₀). SI = Selectivity
Index. Experiments were done in biological triplicates.

| | | | CC₅₀ | EC₅₀ | |
|---|---|---|---|---|---|
| cell type | virus | compound | [nM] | [nM] | SI |
| MRC-5 | HCoV-229E | Silvestrol | >10000 | 3 | >3300 |
| | | CR-31-B (−) | >5000 | 2.88 | >1736 |
| | MERS-CoV | Silvestrol | >10000 | 1.3 | >7690 |
| | | CR-31-B (−) | >5000 | 1.87 | >2674 |
| murine | LASV | Silvestrol | >5000 | 50.73 | >99 |
| hepatocytes | | CR-31-B (−) | >5000 | 36.11 | >139 |
| | CCHFV | Silvestrol | >5000 | 28.53 | >175 |
| | | CR-31-B (−) | >5000 | 20.04 | >250 |
| A549 | ZIKV | Silvestrol | 9.42 | 1.08 | 8.8 |
| | | CR-31-B (−) | 19.34 | 1.13 | 17.1 |

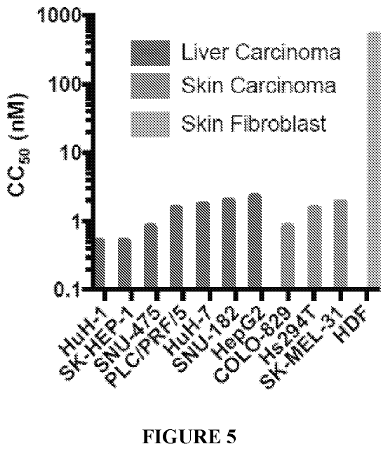

Even if the cells were treated for extended periods of time (48 and 72 h) using concentrations of up to 1 μM, no major cytotoxic effects were observed for CR-31-B (FIG. 4C). We further determined the CC₅₀ values of a racemic mixture of CR-31-B using a range of human liver and skin carcinoma cell lines, as well as primary human dermal fibroblasts (HDF). As expected, CC₅₀ values in all analyzed cancer cell lines (7 liver and 3 skin carcinoma cell lines were found to be in the low nanomolar range, whereas the CC₅₀ in primary HDF was calculated at approximately 500 nM (see Table 5; FIG. 5). Taken together, the data demonstrate low cytotoxicity of CR-31-B (−) in primary cells compared to the known cytotoxic effects in fast growing cancer cell lines.

TABLE 5
CC₅₀ values [nM] determined for the indicated cell types
(human liver and skin carcinoma lines and human dermal fibroblast
(HDF) cells after treatment with racemic (+/−) CR-31-B for
48 h. Data is representative of four experimental replicates.
Average CC50 across the two biological replicates is plotted in FIG. 5.

| | Liver Carcinoma | Skin Carcinoma | Skin Fibroblast |
|---|---|---|---|
| HuH-1 | 0.5 | | |
| SK-HEP-1 | 0.5 | | |
| SNU-475 | 0.8 | | |
| PLC/PRF/5 | 1.5 | | |
| HuH-7 | 1.7 | | |
| SNU-182 | 1.9 | | |
| HepG2 | 2.2 | | |
| COLO-829 | | 0.8 | |
| Hs294T | | 1.5 | |
| SK-MEL-31 | | 1.8 | |
| HDF | | | 500 |

### Example 2: Antiviral Activity of Silvestrol and CR-31-B (−) in a Human Bronchial Epithelial Cell System

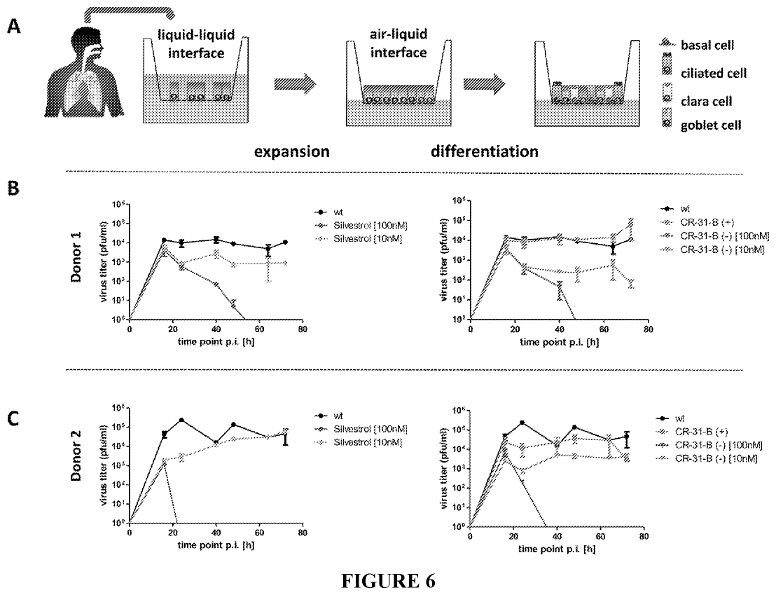

For further evaluation of the antiviral potential of Silvestrol and CR-31-B (−) in a relevant ex vivo system for respiratory viruses, we used primary human airway epithelial cell cultures. Normal human airway epithelial (NHBE) cells were differentiated under air/liquid conditions to pseudostratified (columnar) epithelia. The epithelium was then infected with HCoV-229E to mimic viral infections of different airway cell types in human airways in the presence of inhibitor or solvent control (FIG. 6A). At a concentration of 10 nM, CR-31-B (−) reduced the virus titer in the supernatant by about 1.5 orders of magnitude using cells from two different donors, which was similar to the antiviral effect observed for 10 nM Silvestrol in this ex vivo model. At 100 nM, both compounds reduced infectious virus production to undetectable levels (FIGS. 6B, 6C), whereas 100 nM CR-31-B (+) did not significantly reduce viral replication.

### Example 3: Comparison of Broad-Spectrum Antiviral Activities of Silvestrol and CR-31-B (−)

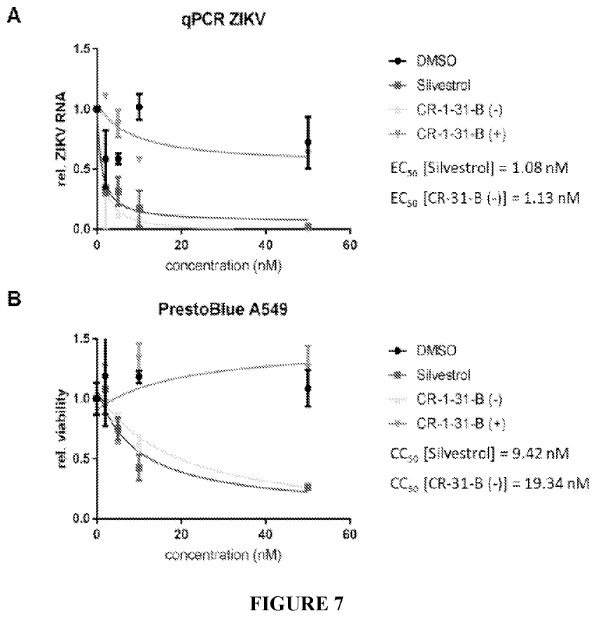

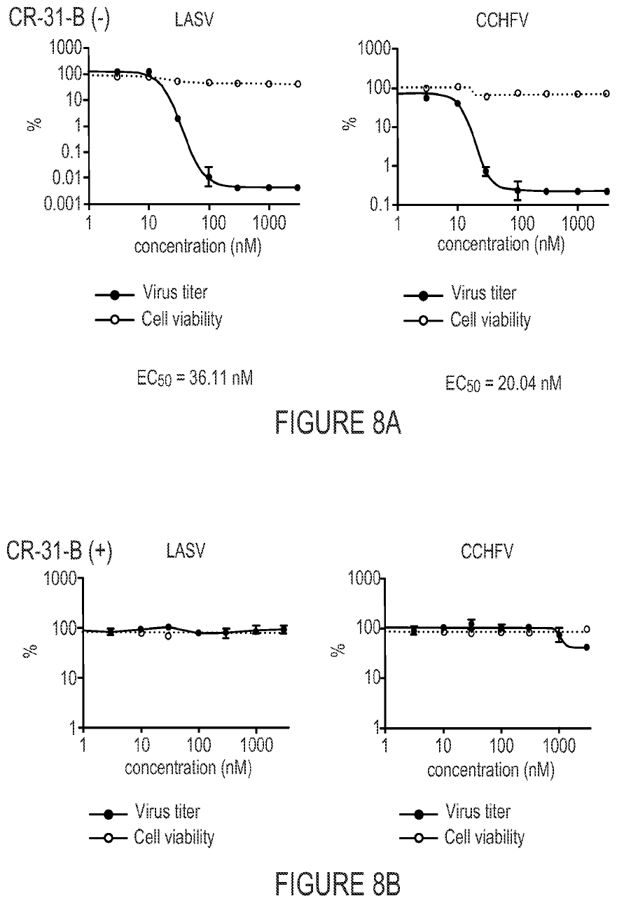

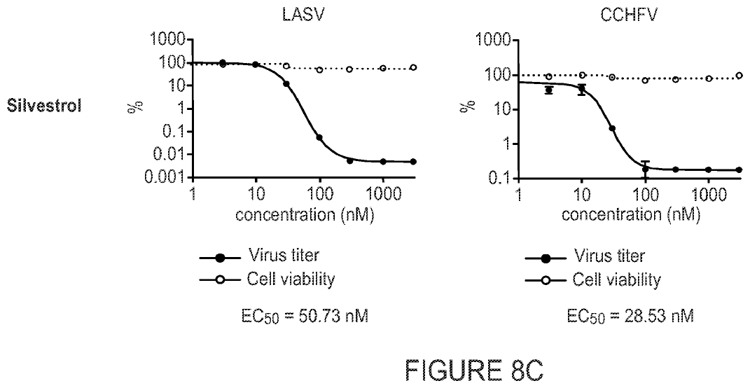

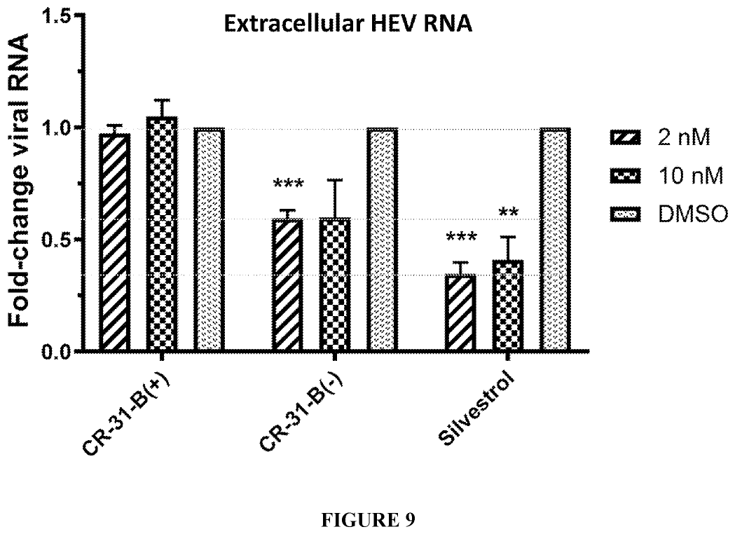

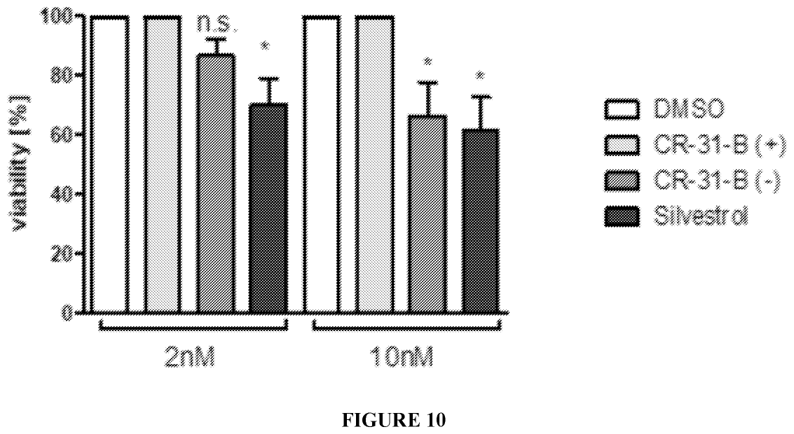

We have recently shown that Silvestrol inhibits Zika virus (ZIKV) replication in the human epithelial lung cell line A549 and in primary human hepatocytes (Elgner et al. [2018] pii. Viruses 10: E149). In the present study, we found that CR-31-B (−) also causes a strong reduction of ZIKV RNA levels with a calculated EC₅₀ value of 1.13 nM (Table 4; FIG. 7). To assess potential broad-spectrum antiviral activities of CR-31-B (−) and Silvestrol against other highly pathogenic emerging viruses, we analyzed their effects in primary murine hepatocytes infected with LASV or CCHFV (FIGS. 8A-8C). The data revealed that CR-31-B (−) and Silvestrol have potent antiviral activities with EC₅₀ values between approximately 20 and 50 nM with no detectable cytotoxicity at concentrations of up to 10 μM (FIGS. 8A-8C). Virus titers showed an approximately 4-log drop for LASV- and a 3-log drop for CCHFV-infected cells (FIGS. 8A, 8C). Furthermore, we analyzed whether CR-31-B (−) inhibits HEV replication (FIG. 9). Persistently HEV-infected A549 cells were treated with different concentrations of Silvestrol and the two CR-31-B enantiomers. At 72 hpi the extracellular viral RNA levels, which correlate with released viral particles, were analyzed using quantitative RT-PCR. In line with our previous results (Glitscher et al. [2018] pii. Viruses 10: E301), we confirmed the antiviral activity of Silvestrol, while the antiviral effect of CR-31-B (−) against HEV was found to be slightly weaker at the low nanomolar concentrations used in this assay (FIG. 9). However, some cytotoxicity of Silvestrol was already observed at a concentration of 2 nM, whereas CR-31-B was not cytotoxic at this concentration, yet at 10 nM some cytotoxicity of CR-31-B (−) could be observed (approximately 35% reduced viability) in persistently HEV-infected A549 cells (FIG. 10).

### Example 4: Analyses of the 5′-UTR-Mediated Inhibitory Activities of Silvestrol and CR-31-B (−)

To gain more mechanistic insights regarding the effects of Silvestrol and CR-31-B (−) on translation initiation and RNA clamping, we compared the inhibitory effects of the two rocaglates on different viral 5′-UTRs in a dual luciferase reporter assay (FIG. 11; Müller et al. [2018] Antivir. Res. 150: 123-129).

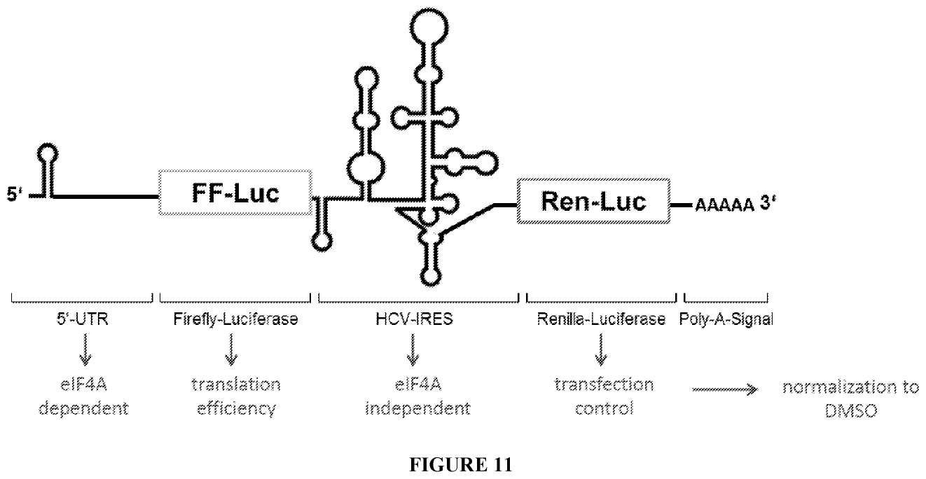

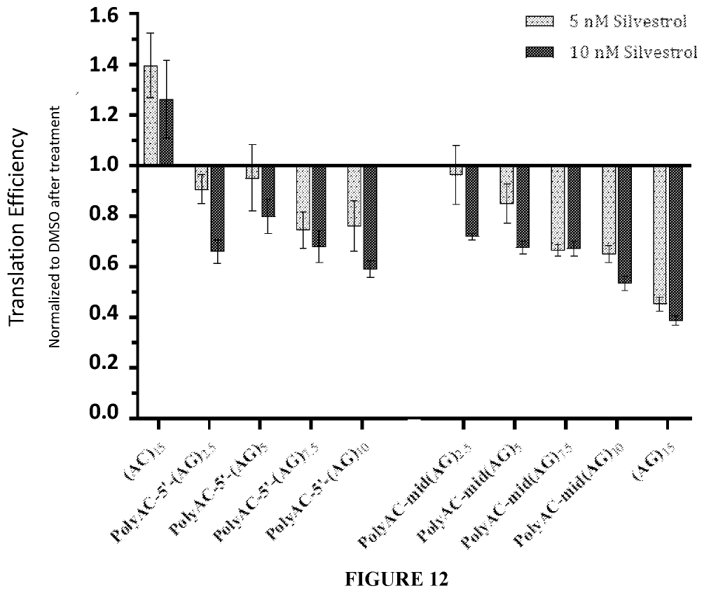

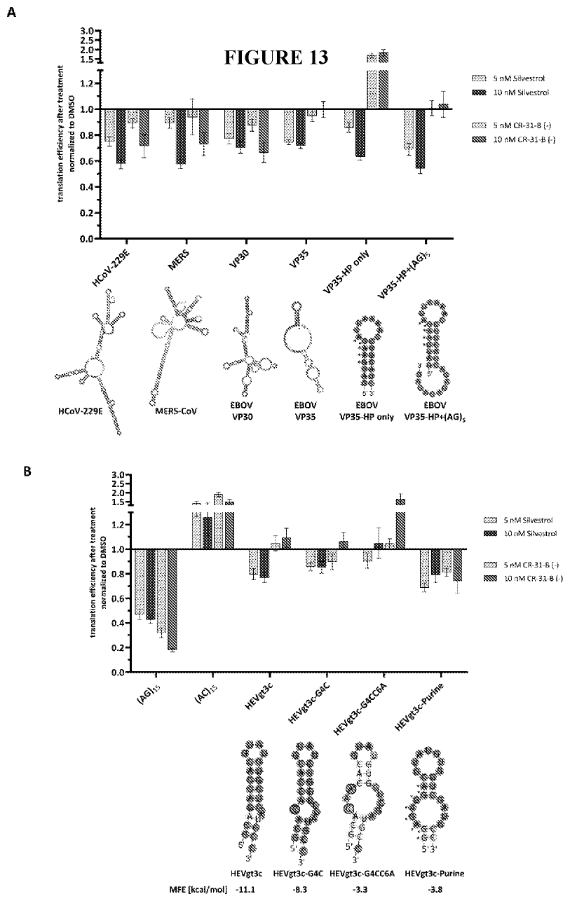

We began by examining the effects of Silvestrol on the translation efficiency of reporter gene expression constructs containing various artificial 5′-UTRs (see Table 1). We demonstrated that 5 purines in a row embedded in a pyrimidine sequence can induce rocaglate sensitivity (polyAC-5′-(AG)_{2.5}) (FIG. 12). We have shown that the viral 5′UTR from hepatis E virus, where no polypurine stretch is present (HEVgt3c), is not sensitive towards CR-31-B (−) treatment (Müller et al. [2020] Antivir. Res. 175: 104706). By introducing a polypurine stretch of 5 nt in length (HEVgt3c-Purine), CR-31-B (−) sensitivity was introduced (FIG. 13B). From this, it appears that five (5) consecutive purines are the minimum for RNA clamping.

The 5′-UTRs of HCoV-229E, MERS-CoV, and EBOV VP30 mRNA were found to be similarly sensitive to translation inhibition by Silvestrol and CR-31-B (−) (FIG. 13A). Interestingly, the VP30 and VP35 mRNAs both carry a likely inaccessible pentapurine stretch in their 5′-terminal stem structures (FIG. 13A). However, in the VP30 5′-UTR, this pentapurine stretch is followed by a decapurine stretch that seems to be unstructured and thus predicted to enable RNA clamping (Iwasaki et al. [2019] Mol. Cell 73: 738-748 e9). Such a second purine stretch is absent in the VP35 5′-UTR. The 5′-terminal alone is sufficient to mediate translation inhibition by Silvestrol. Surprisingly, this hairpin increased the reporter activity about twofold in the presence of CR-31-B (−) (FIG. 13A, VP35-HP only). This increase was reduced back to basic levels if an additional (AG)₅-polypurine stretch was inserted at the 3′-end of the VP35 hairpin. Also, this insertion resulted in a slightly increased translation inhibition by Silvestrol, indicating that polypurine sequences strengthen the inhibitory effects of rocaglates on eIF4A (FIG. 13A, VP35-HP+(AG)₅).

To confirm the idea of polypurines being required for stable stacking interactions with rocaglates, we constructed 5′-UTRs consisting of a 30-nt long (AG)₁₅ or an unstructured (AC)₁₅ sequences as negative control. As presumed, Silvestrol inhibited translation of the (AG)₁₅ construct by approximately 55% and CR-31-B (−) even by approximately 80% at concentrations of 10 nM (FIG. 13B). Remarkably, the presence of the (AC)₁₅ sequence caused a 1.5-fold induction of luciferase activity with both compounds (FIG. 13B), indicating that the helicase activity of eIF4A was dispensable in this case.

Interestingly, the 5′-UTR of HEV (HEVgt3c) lacks any polypurine sequence element, but it is predicted to form a stable RNA hairpin structure which, most likely, requires unwinding during translation initiation. As shown before, HEV replication can be inhibited by Silvestrol (Glitscher et al. [2018] pii. Viruses 10: E301) and, to a slightly less extent, by CR-31-B (−) (FIG. 9). Therefore, we asked if the polypurine-free 5′-UTR of HEV is also sensitive towards Silvestrol or CR-31-B (−) treatment. At a concentration of 10 nM Silvestrol, the 5′-UTR of HEV mediated reduced luciferase activity, demonstrating that Silvestrol can indeed clamp this viral RNA onto eIF4A. Importantly, this was not the case for CR-31-B (−) treatment (FIG. 13B, HEVgt3c). Moreover, this indicated that the dioxane moiety of Silvestrol may play a critical role in clamping structured RNAs onto eIF4A in a polypurine-independent manner. To test this assumption, the HEV 5′-UTR hairpin structure was thermodynamically destabilized by disrupting one or two base pairs in the stem (FIG. 13B, HEVgt3c and HEVgt3C-G4CC6A). These changes led to a gradual loss of the inhibitory effect of Silvestrol. To further analyze the relevance of polypurine stretches in viral 5′-UTRs, the sequence in the HEV 5′-UTR was changed at the 5′-end from 5′-GCAGACCA . . . (SEQ ID NO: 34) into 5′-GGAGAGGA . . . (SEQ ID NO: 35) (FIG. 13B, HEVgt3c-Purine), thereby introducing a stretch of 8 consecutive purines. Although the thermodynamic stability of the hairpin structure was reduced by these sequence changes, the HEVgt3c-Purine 5′-UTR became sensitive now to CR-31-B (−) and Silvestrol treatment (FIG. 13B, HEVgt3c-Purine).

### Example 5: Modelling of Silvestrol onto the Surface Structure of the Human eIF4A-Polypurine RNA Complex

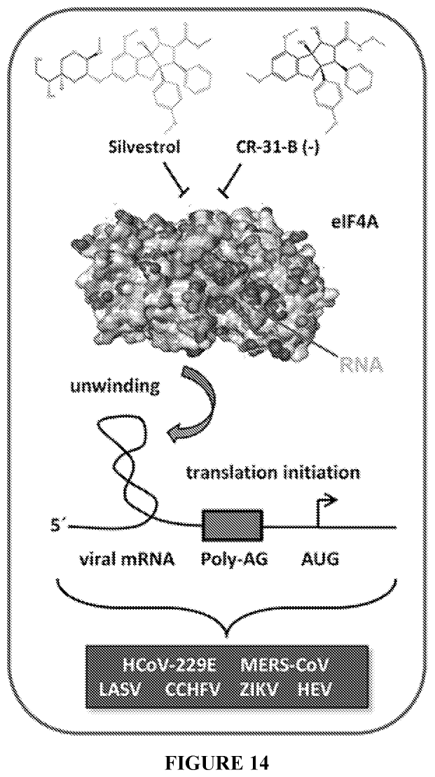

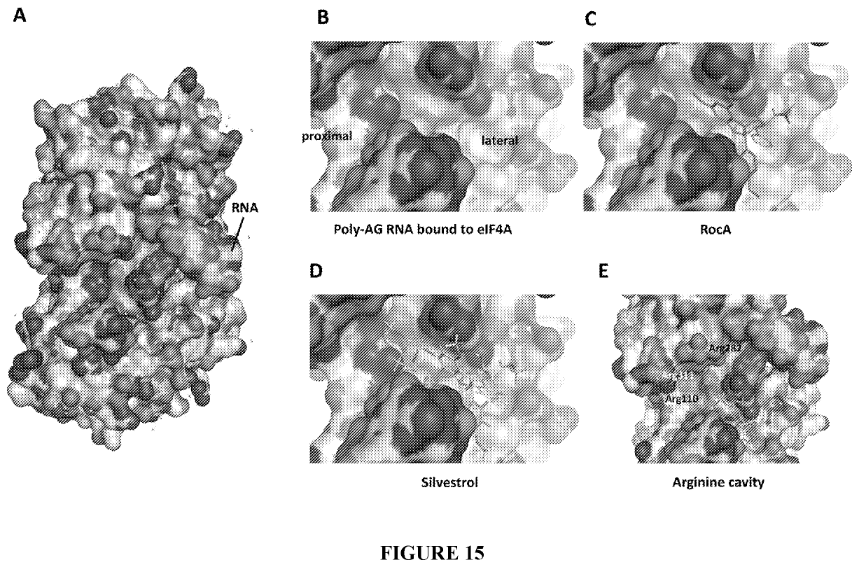

Our reporter assay results indicated differences in the mode of action between Silvestrol and rocaglates lacking the dioxane moiety. Since no structural data from co-crystallization of Silvestrol, RNA and eIF4A are available, we modeled the structure of Silvestrol into the published eIF4A-polyAG structure surface (Iwasaki et al. [2019] Mol. Cell 73: 738-748 e9); see also FIG. 14). By comparing the localization of RocA and Silvestrol, we found that the dioxane moiety of Silvestrol can completely cross the surface of the bound RNA substrate thus enabling Silvestrol to make additional contacts, e.g., via H-bonds to arginines in eIF4A which cannot be formed in the absence of the dioxane moiety (FIG. 15). This suggests that Silvestrol is able to clamp RNA without an absolute requirement for stacking interactions with purine bases because stabilizing interactions with eIF4A at the proximal side of the bound RNA may be sufficient for locking the RNA on the helicase.

### Example 6: Inhibitory Effect of CR-31-B (−) on eIF4A-Dependent Translation of Viral 5′-UTRs

We analyzed the potential inhibitory effect of CR-31-B (−) (FIG. 1) on 5′-UTRs of different coronaviruses including SARS-CoV-2 in a dual luciferase reporter assay (FIG. 11) to assess whether translation of mRNAs containing these viral 5′-UTRs depended on eIF4A.

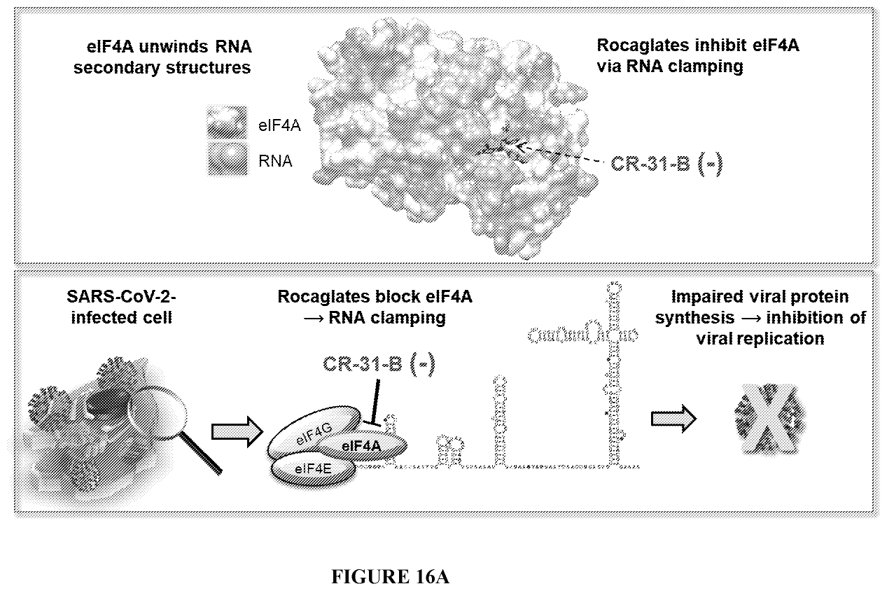

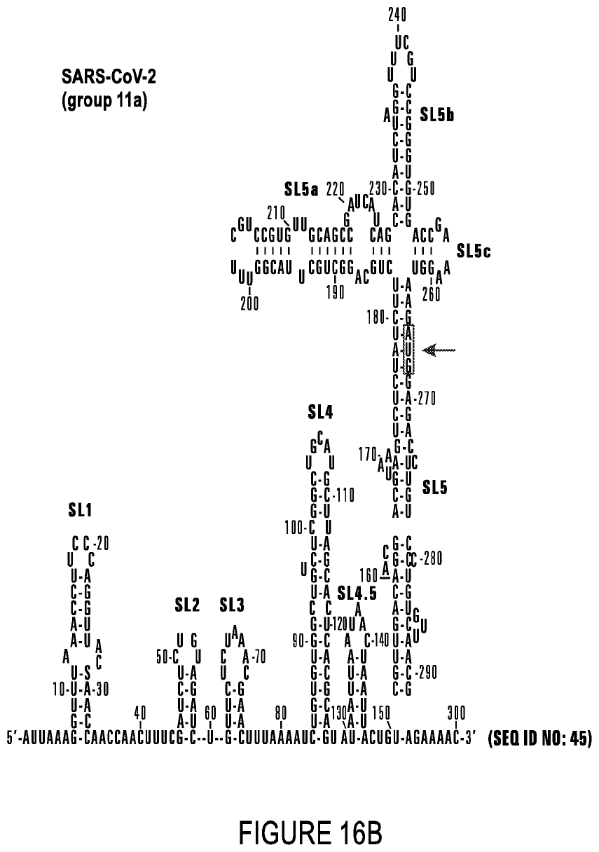

In one non-limiting example with respect to SARS-CoV-2, FIG. 16A (upper panel) depicts a model for the predicted binding mode of CR-31-B (−) on a human eIF4A-RNA complex. The RNA binds via its negative phosphate backbone onto eIF4A in a cavity formed by the two domains of the helicase that mainly consists of positively charged amino acids. With respect to Silvestrol, the arginines, which have a positive charge, can be reached by the dioxane moiety on the left side of the bound RNA. With respect to CR-31-B (−), this is not particularly relevant. White indicates neutral amino acids (no charge or polar groups in the side chain). Binding of CR-31-B (−) leads to RNA clamping shown by structure-based comparative modeling. FIG. 16A (lower panel) is a schematic depicting SARS-CoV-2 infected cells, the predicted secondary structure of the SARS-CoV-2 5′-UTR with the 5′-cap bound translation initiation complex eIF4F (consisting of the cap-binding protein eIF4E, the bridging protein eIF4G and the DEAD-box RNA helicase eIF4A). RNA clamping of CR-31-B (−) blocks translation and strongly reduces viral protein synthesis and as a consequence viral replication. FIG. 16B is a schematic depicting the RNA sequence and predicted secondary structure of the SARS-CoV-2 5′UTR (identical with Group IIa) (see SEQ ID NO: 45; Table 6). The sequence ends directly before the start codon AUG (marked in yellow) ( . . . AAG AUG . . . ), is located between SL5c and SL5 (SL means “stem loop”), and is shown in Table 6 (above).

Cap-dependent translation of the firefly luciferase gene was measured and transfection efficiencies were normalized to renilla luciferase expression, which is under control of an eIF4A-independent internal ribosome entry site (IRES) element from hepatitis C virus (HCV) (FIG. 11). The short and unstructured 5′-UTR of the beta-globin gene and an unstructured (AC)₁₅ sequence served as negative controls, since these 5′-UTR sequences were shown to be not repressible upon inhibition of eIF4A eIF4A (Müller et al.

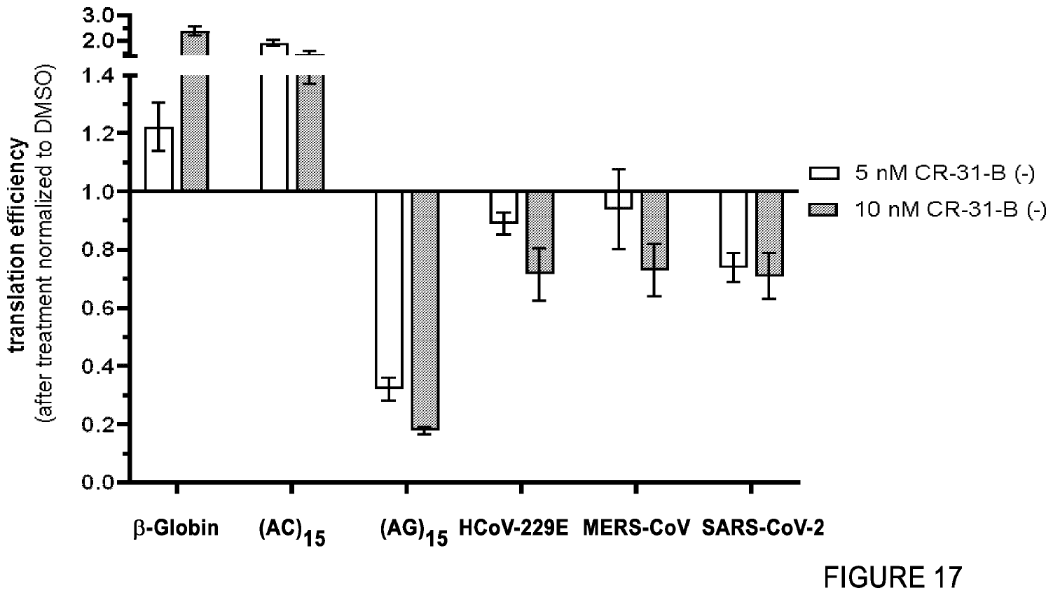

Antiviral Res. 150: 123-129; Müller et al. [2020] Antiviral. Res. 175: 104706). The polypurine sequence (AG)₁₅ was used as a positive control since this sequence can be efficiently clamped onto the surface of eIF4A by different rocaglates due to π-π (pi-pi) stacking interactions (Müller et al. [2020] Antiviral. Res. 175: 104706; Iwasaki et al. [2019] Mol. Cell 73: 738-748 e9). Using this experimental setup, eIF4A-dependency was inferred from sensitivity of firefly luciferase mRNA translation to the presence of a specific eIF4A inhibitor. The data revealed that the 5′-UTRs of SARS-CoV-2, HCoV-229E and MERS-CoV were similarly sensitive to eIF4A-dependent translation inhibition by CR-31-B (−) in the dual luciferase reporter assay, when 10 nM CR-31-B (−) was used (FIG. 17), indicating that CR-31-B (−) may have antiviral activity against the newly emerging SARS-CoV-2.

### Example 7: In Vitro Antiviral Effect of CR-31-B (−) Against SARS-CoV-2 in African Green Monkey Vero E6 Cells

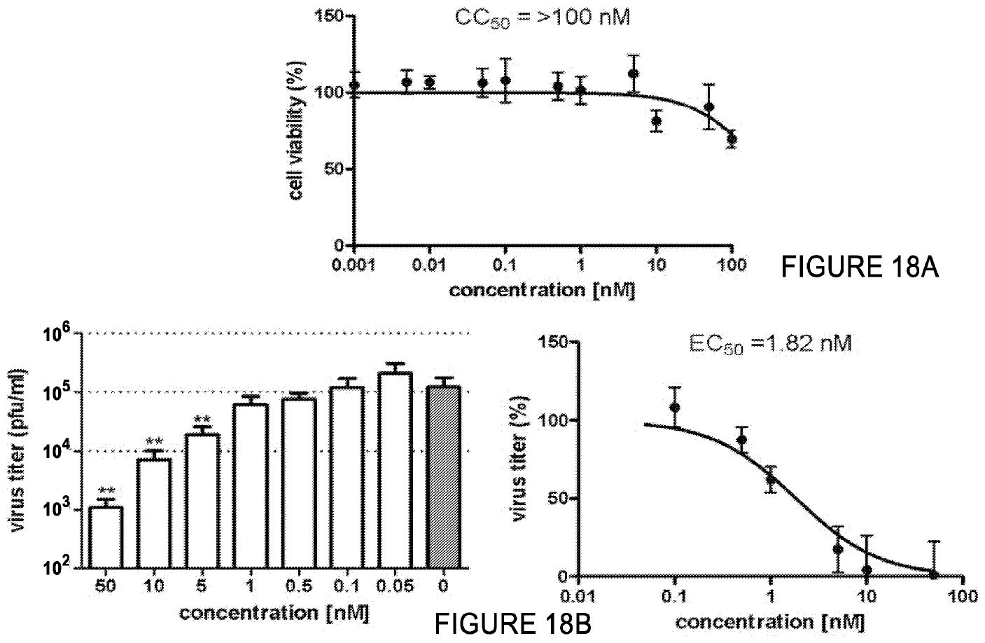

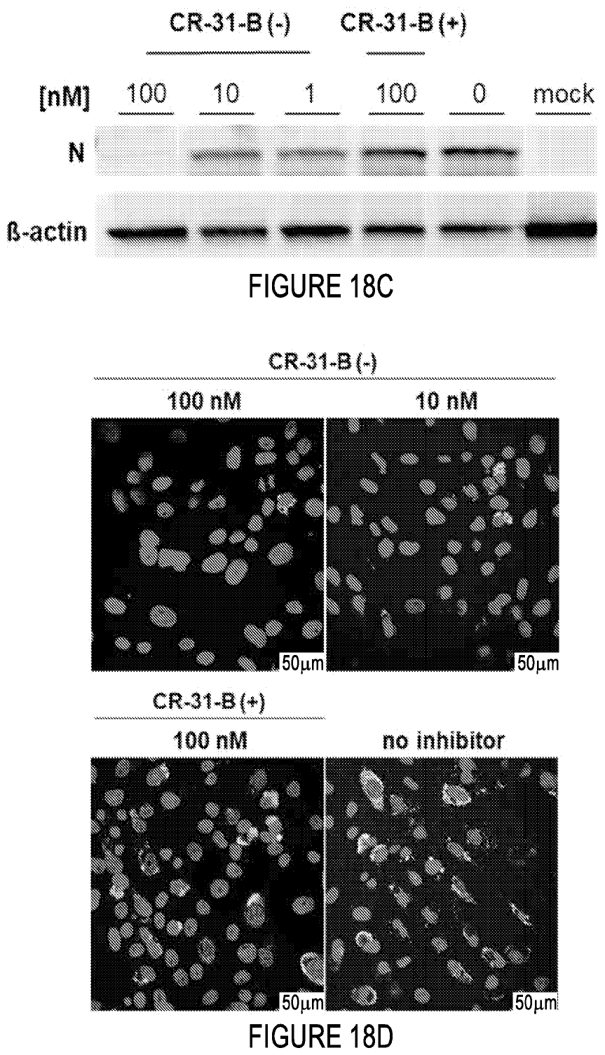

To analyze possible antiviral effects of CR-31-B (−) in cell culture, African green monkey Vero E6 cells were used (Ogando et al. [2020]J. Gen. Virol. doi: 10.1099/jgv.0.001453). Cytotoxicity of CR-31-B (−) was determined by MTT assay by treating Vero E6 cells with increasing concentrations of CR-31-B (−) for 24 h. No major cytotoxicity was detected for concentrations of up to 100 nM, with cell viability being reduced by about 10-25% at the highest concentration tested (FIG. 18A). To determine antiviral activity of CR-31-B (−) against SARS-CoV-2, Vero E6 cells were infected with this virus at an MOI of 0.1 plaque forming units (pfu)/cell and incubated with medium containing the different concentrations of CR-31-B (−). At 24 h p.i., cell culture supernatants were collected, and virus titers were determined by plaque assay. Moreover, the production of infectious SARS-CoV-2 progeny was found to be reduced in a dose-dependent manner with an EC₅₀ of approximately 1.8 nM (˜1.8 nM) (FIG. 18B), which is in a similar range with the CR-31-B (−) EC₅₀ values reported previously for other coronaviruses (2.88 nM for HCoV-229E; 1.87 nM for MERS-CoV) (Müller et al. [2020] Antiviral Res. 175: 104706). The selectivity index (CC₅₀/EC₅₀) for SARS-CoV-2-infected Vero E6 cells was determined to be >50. Next, we analyzed the effect of CR-31-B (−) on SARS-CoV-2 protein accumulation and the formation of viral replication/transcription complexes in Vero E6 cells. Viral nucleocapsid (N) protein levels were found to be drastically reduced in the presence of 100 nM CR-31-B (−), and moderately reduced at concentrations of 10 nM or 1 nM (FIG. 18C). As expected, viral protein accumulation was not affected in the presence of 100 nM of the inactive (+)-enantiomer CR-31-B (+) nor was it affected in cells treated with solvent control only.

In line with this, the formation of SARS-CoV-2 replication/transcription complexes was impaired in infected cells treated with CR-31-B (−) (FIG. 18D). As shown in FIG. 18D, immunofluorescence analysis using antibodies specific for double-stranded RNA (dsRNA) (a mouse anti-dsRNA mAb (J2, SCICONS English & Scientific Consulting Kft.), representing a viral RNA replication intermediate, revealed a profound reduction of replicative organelles active in viral RNA synthesis (Müller et al. [2018a]J. Virol. 92: :(4): e01463-17. doi: 10.1128/JVI.01463-17).

### Example 8: Antiviral Activity of CR-31-B (−) and Silvestrol Against SARS-CoV-2 in an Ex Vivo Human Bronchial Epithelial Cell System

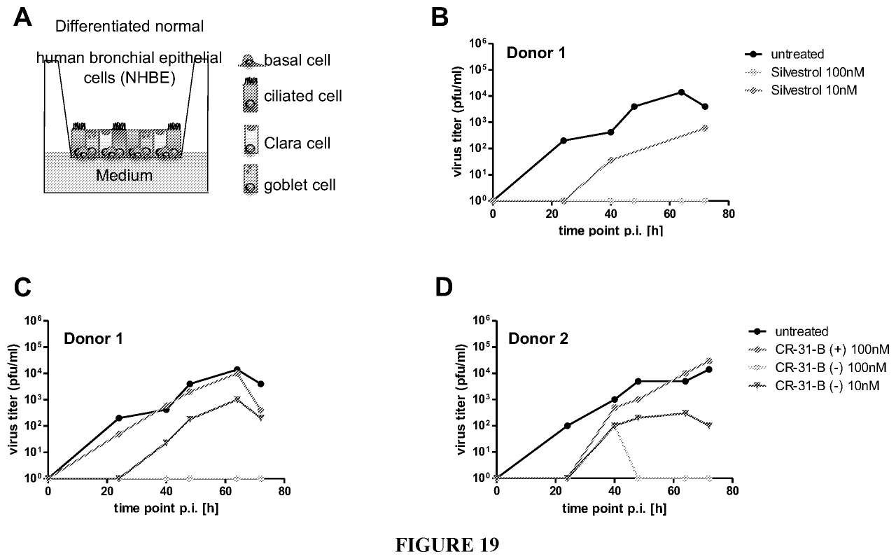

To further evaluate the antiviral potential of CR-31-B (−) in a biologically relevant ex vivo respiratory cell culture system, we analyzed air/liquid interface (ALI) cultures of differentiated primary normal human bronchial epithelial (NHBE) cells isolated from two different donors. ALI cultures are increasingly recognized as an excellent culture model mimicking the tracheobronchial region of the human respiratory tract and thus enabling respiratory infection research in physiologically relevant cellular environment (Jonsdottir et al. [2016] Virol. J. 13:24). Differentiated NHBE cells (FIG. 19A) were infected with SARS-CoV-2 (MOI=3 pfu/cell) in the presence of inhibitor, CR-31-B (−) or Silvestrol (obtained from the Sarawak Biodiversity Centre, Kuching; North-Borneo, Malaysia; purity >99%), the inactive enantiomer CR-31-B (+) or solvent control (untreated). Silvestrol was included as a reference in this experiment because this rocaglate has previously been tested extensively against a broad range of viruses. Silvestrol treatment reduced SARS-CoV-2 titers at different time points p.i. about 10- to 100-fold when used at a concentration of 10 nM, while virus replication in NHBE cells was completely abolished at a concentration of 100 nM (FIG. 19B). Next, we analyzed the effects of CR-31-B in NHBE cells. CR-31-B (−) reduced the production of infectious virus progeny by approximately 1.5 (˜1.5) log steps at a concentration of 10 nM in differentiated NHBE cells obtained from two different donors. At 100 nM, CR-31-B (−) reduced SARS-CoV-2 titers to undetectable levels, whereas the inactive enantiomer CR-31-B (+) did not affect viral replication compared to the solvent control (FIGS. 19C, 19D). No obvious cytotoxicity could be observed at this concentration using light microscopy.

Taken together, the data confirm a potent antiviral activity of CR-31-B (−) against this newly emerging coronavirus in a human ex vivo cell culture system. The compound proved to be active at nanomolar concentrations, similar to those demonstrated for, e.g., HCoV-229E.

### DISCUSSION

The ongoing SARS-CoV-2 pandemic, a coronavirus outbreak of zoonotic origin reminiscent of the SARS and MERS outbreaks a few years earlier, has led to an increased awareness of the need for first-line, broad-spectrum ‘pan-antivirals’ that can be used as stopgaps until vaccines and specific therapies become available to prevent or treat infections caused by newly emerging viruses. We have focused on the discovery and development of antivirals that target host mechanisms critical to viral proliferation. The rationale behind this approach is twofold: first, host mechanisms are not virus-specific but are used by a broad range of viruses, and second, targeting a host mechanism preempts the risk for developing resistance typically associated with targeting viral structures or mechanisms.

We have identified the synthetic rocaglate CR-31-B (−) as a novel potent antiviral compound with broad-spectrum activity against HCoV-229E, MERS-CoV, LASV, CCHFV, HEV, and ZIKV. The broad-spectrum activity against these various RNA viruses clearly could apply to other RNA viruses. Moreover, the antiviral potential of Silvestrol and CR-31-B (−) was compared in ex vivo human airway epithelial cell system under air/liquid conditions. These differentiated airway epithelial cells were used as a model for the primary airway defense barrier against inhaled pathogens, mimicking the natural situation in the infected host. This system provides a pseudostratified organization of basal, ciliated, goblet, and other less common types of cells and plays a crucial role in maintaining airway homeostasis by regulating innate and acquired immunity through the production of a wide range of cytokines as well as chemokines (Davies DE [2014] Ann. Am. Thorac. Soc. 11 (Supp. 5): S224-S251). In this relevant ex vivo cell system, we could confirm the antiviral potential of CR-31-B (−), as well as Silvestrol, on HCoV-229E replication.

CR-31-B (−) has a potent antiviral activity similar to that of the more complex-structured Silvestrol. It is active at low nanomolar concentrations with low cytotoxicity in primary human cells, while it has a higher cytotoxicity in cancer cell lines (see FIG. 5). Thus, CR-31-B (−) opens a broad therapeutic window for the treatment of viral infections and qualifies as an interesting synthetic rocaglate for further in vivo evaluations. Even though the antiviral potential of CR-31-B (−) is similar to Silvestrol, we identified substantial mechanistically differences between the two compounds as detailed below.

RNAs generally bind to eIF4A in a sequence- and structure-independent manner via their phosphate backbone. Thus, if no RNA clamping by rocaglates occurs, active eIF4A retains its ability to unwind secondary structures in the bound substrate RNA. In our reporter assays, Silvestrol was able to clamp polypurine-free stable hairpin structures onto eIF4A, whereas CR-31-B (−) required an accessible polypurine-sequence in proximity to the 5′-terminal hairpin. This conclusion is supported by data showing that translation of the reporter construct containing the EBOV VP35 5′-UTR is insensitive to CR-31-B (−), while it is sensitive to Silvestrol. Reduced hairpin stability correlated with a loss in Silvestrol sensitivity, suggesting that thermodynamic stability of hairpin structures is of critical importance. This is also supported by our observation that introduction of an unstructured (AC)₁₅ sequence rendered the reporter construct insensitive to both Silvestrol and CR-31-B (−), whereas an (AG)₁₅ polypurine sequence, which might be able to form a stable hairpin structure by making non-Watson-Crick G-A base pair contacts, strongly requires the helicase activity of eIF4A. Remodeling the structure of Silvestrol onto the surface of the published eIF4A-RNA structure revealed that Silvestrol is in principle capable to contact eIF4A via phenylalanine (Phe163) at the lateral (Iwasaki et al. [2019] Mol Cell 73: 738-748 e9) and via arginines at the proximal site of the bound substrate RNA (FIG. 14 and FIG. 15). This leads us to suggest that Silvestrol may be able to clamp any RNA sequence onto eIF4A. Our results (FIG. 13B) indicate that an unstructured sequence of sufficient length, e.g., the (AC)₁₅ sequence, does not require the helicase activity of eIF4A to allow 43S PIC binding as a prerequisite for translation initiation, whereas a stable hairpin structure likely prevents 43S PIC binding and thus needs to be unwound.

The published crystal structure of human eIF4A together with a polypurine RNA substrate and RocA (Iwasaki et al. [2019] Mol Cell 73: 738-748 e9) helps to explain why polypurine sequences are required to clamp RNA onto eIF4A by RocA. Stacking interactions between two phenyl rings of RocA and two consecutive purines in the substrate RNA are stable enough for RNA clamping onto eIF4A, whereas less stable interactions with pyrimidines are insufficient (Iwasaki et al. [2019] Mol Cell 73: 738-748 e9). As a consequence, a stable hairpin structure without a polypurine stretch may not be bound efficiently by RocA or CR-31-B (−) due to the lack of the dioxane moiety. To date, there is no structural information on the exact binding mode of Silvestrol onto eIF4A. We therefore initiated co-crystallization studies with human eIF4A, poly-AG RNA, and Silvestrol to identify the exact position of the dioxane moiety.

Surprisingly, we observed some antiviral activity of CR-31-B (−) in HEV-producing A549 cells at a non-cytotoxic concentration of 2 nM even though the compound did not inhibit reporter translation in the presence of a polypurine-free HEVgt3c 5′-UTR. We propose an indirect effect of CR-31-B (−) that could be mediated by eIF4A-dependent cellular mRNAs, e.g., of unknown identity.

Importantly, the effects of CR-31-B in vivo are comparable to Silvestrol. In a xenograft mouse model, both compounds reduced tumor growth efficiently (Wolfe et al. Nature 513: 65-70). CR-31-B treatment did not change body weight, or the number of blood cells and no toxicity could be observed in the gastrointestinal tract of mice. Moreover, serum levels of aminotransferases (ALT and AST), albumin, total bilirubin, as well as creatinine, were not significantly changed two weeks after cessation of treatment, indicating that CR-31-B has a favorable toxicity profile in vivo (Wolfe et al. [2014] Nature 513: 65-70). So far, no published data on the pharmacokinetic properties of CR-31-B (−) are available. It was already shown that daily intraperitonealinjection of CR-31-B (−) works well in mice (Wolfe et al. [2014] Nature 513: 65-70; Chan et al. [2019] Nat. Commun. 10: 5151), while the systemic availability of rocaglates following oral application seems to be limited (Saradhi et al. [2011] AAPS J. 13: 347-356). Based on this and our own data, we conclude that CR-31-B (−) may represent an interesting alternative to the broad-spectrum antiviral Silvestrol, which remains to be confirmed in appropriate in vivo studies to evaluate and compare the antiviral potential, toxicity, and pharmacokinetics profiles of the two compounds.

Our results confirm that CR-31-B (−) has potent broad-spectrum antiviral activity similar to that of Silvestrol (Table 4). Moreover, polypurine sequences in the 5′-UTR were found to be required for rocaglate-dependent clamping onto eIF4A if the dioxane moiety of Silvestrol is missing as shown for the synthetic CR-31-B (−) compound. Our data also suggest that Silvestrol retains its ability to clamp RNA substrates containing a stable hairpin structure in cases where polypurine sequences are not accessible (e.g., the VP35 5′-UTR) or absent (e.g., the HEV 5′-UTR).

We have also explored the broad-spectrum antiviral strategy of targeting the host factor eIF4A to inhibit SARS-CoV-2 replication by analyzing the antiviral activity of the synthetic rocaglate CR-31-B (−), a specific inhibitor of eIF4A-dependent mRNA translation. We have identified CR-31-B (−) as a potent and non-cytotoxic inhibitor of SARS-CoV-2 replication in vitro and ex vivo. The observed antiviral activities are directly comparable to those we have demonstrated for other coronaviruses, namely HCoV-229E and MERS-CoV, and a range of highly pathogenic positive- and negative-sense single-stranded RNA viruses. We have demonstrated inhibition of SARS-CoV-2 proliferation in a relevant ex vivo human bronchial cell system by a rocaglate.

### SUMMARY

Here, we model how rocaglates form stacking interactions with polypurine sequences in the 5′-untranslated regions (UTRs) of capped mRNAs, clamping the mRNAs onto eIF4A and stalling mRNA unwinding, which results in a dissociation of the mRNA-eIF4A complex from eIF4E and eIF4G, and we present the less complex structured (compared with Silvestrol) synthetic rocaglate CR-31-B (−) as a compound with potent broad-spectrum antiviral activity in primary cells and in an ex vivo bronchial epithelial cell system. CR-31-B (−) inhibits replication of Corona-, Zika-, Lassa-, Crimean Congo hemorrhagic fever viruses and, to a lesser extent, hepatitis E viruses at non-cytotoxic low nanomolar concentrations. Since hepatitis E virus has a polypurine-free 5′-UTR that folds into a stable hairpin structure, we hypothesized that RNA clamping by Silvestrol and its derivatives may also occur in a polypurine-independent but structure-dependent manner. Interestingly, the HEV 5′-UTR conferred sensitivity towards Silvestrol but not to CR-31-B (−). However, if an exposed polypurine stretch was introduced into the HEV 5′-UTR, CR-31-B (−) became an active inhibitor comparable to Silvestrol. Moreover, thermodynamic destabilization of the HEV 5′-UTR led to reduced translational inhibition by Silvestrol, suggesting differences between rocaglates in their mode of action, most probably by engaging Silvestrol's additional dioxane moiety.

In African green monkey Vero E6 cells, CR-31-B (−) inhibited SARS-CoV-2 replication with an EC₅₀ of approximately 1.8 (˜1.8) nM. In line with this, viral protein accumulation and replication/transcription complex formation were found to be strongly reduced by this compound. In an ex vivo infection system using human airway epithelial cells, CR-31-B (−) was found to cause a massive reduction of SARS-CoV-2 titers by about 4 logs to nearly non-detectable levels. The data reveal a potent anti-SARS-CoV-2 activity by CR-31-B (−), corroborating results obtained for other coronaviruses and supporting the concept that rocaglates may be used in first-line antiviral intervention strategies against RNA virus outbreaks.

In summary, the consistent antiviral efficacy of this class of compounds across viruses both in vitro and ex vivo demonstrates that rocaglates represent a powerful addition to the toolbox of interventions available to public health authorities to counter new and emerging RNA virus outbreaks globally.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating a viral infection in a host cell or organism infected by the virus, the method comprising administering to the cell or organism a therapeutically effective amount of a pharmaceutical composition comprising CR-31-B or a pharmaceutically acceptable salt thereof.

2. The method of claim 1, wherein the CR-31-B comprises a racemic mixture of:
(a) a CR-31-B (−) enantiomer having the formula 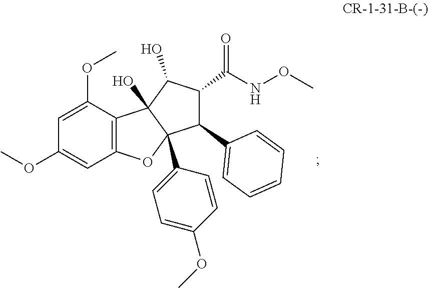
and
(b) a CR-31-B (+) enantiomer having the formula

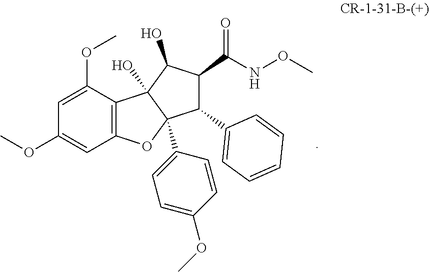

3. The method of claim 1, wherein the CR-31-B comprises at least 50% CR-31-B (−) enantiomer.

4. The method of claim 1, wherein the CR-31-B is a CR-31-B (−) enantiomer.

5. The method of claim 1 or claim 4, wherein the CR-31-B reduces or inhibits a eukaryotic initiation factor 4A (eIF4A) activity, reduces or inhibits a eIF4A helicase activity, reduces or inhibits eIF4A clamping to a 5′-untranslated region (5′-UTR) of the mRNA of the virus, or any combination thereof.

6.-7. (canceled)

8. The method of claim 5, wherein the 5′-UTR comprises a hairpin structure.

9. The method of claim 5, wherein the 5′-UTR comprises a polypurine sequence element comprising at least 5 purine nucleotides.

10. The method of claim 9, wherein the polypurine sequence element comprises at least 20 purine nucleotides.

11. The method of claim 1, wherein the virus comprises a virus from the Coronaviridae family, the Arenaviridae family, the Nairoviridae family, the Flaviviridae family, the Hepeviridae family, the Filoviridae family, or the Togaviridae family.

12. The method of claim 11, wherein the virus from the Coronaviridae family comprises severe acute respiratory syndrome coronavirus 2 (SARS-CoV-2, COVID-19 virus), severe acute respiratory syndrome coronavirus (SARS-CoV), Middle East respiratory syndrome coronavirus (MERS-CoV), human coronavirus 229E (HCoV-229E), human coronavirus OC43 (HCoV-OC43), human coronavirus NL63 (HCoV-NL63), or human coronavirus HKU1 (HCoV-HKU1).

13.-19. (canceled)

20. The method of claim 1, wherein the composition further comprises a pharmaceutically acceptable carrier, excipient, or diluent.

21. A method for reducing or inhibiting translation initiation of a messenger ribonucleic acid (mRNA) of a virus in a host cell or organism infected by the virus, the method comprising administering to the cell or organism a therapeutically effective amount of a pharmaceutical composition comprising CR-31-B or a pharmaceutically acceptable salt thereof.

22. The method of claim 21, wherein the CR-31-B comprises a racemic mixture of:
(a) a CR-31-B (−) enantiomer having the formula 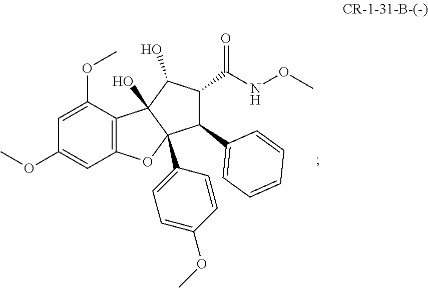
and
(b) a CR-31-B (+) enantiomer having the formula

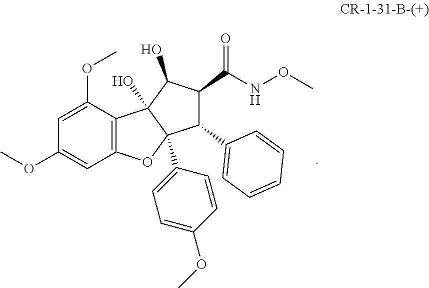

23. The method of claim 21, wherein the CR-31-B comprises at least 50% CR-31-B (−) enantiomer.

24. The method of claim 21, wherein the CR-31-B is a CR-31-B (−) enantiomer.

25. The method of claim 21, wherein the CR-31-B reduces or inhibits a eukaryotic initiation factor 4A (eIF4A) activity, reduces or inhibits a eIF4A helicase activity, reduces or inhibits eIF4A clamping to a 5′-untranslated region (5′-UTR) of the mRNA of the virus, or any combination thereof.

26.-27. (canceled)

28. The method of claim 25, wherein the 5′-UTR comprises a hairpin structure.

29. The method of claim 25, wherein the 5′-UTR comprises a polypurine sequence element comprising at least 5 purine nucleotides.

30. The method of claim 29, wherein the polypurine sequence element comprises at least 20 purine nucleotides.

31. The method of claim 21, wherein the virus comprises a virus from the Coronaviridae family, the Arenaviridae family, the Nairoviridae family, the Flaviviridae family, the Hepeviridae family, the Filoviridae family, or the Togaviridae family.

32. The method of claim 31, wherein the virus from the Coronaviridae family comprises severe acute respiratory syndrome coronavirus 2 (SARS-CoV-2, COVID-19 virus), severe acute respiratory syndrome coronavirus (SARS-CoV), Middle East respiratory syndrome coronavirus (MERS-CoV), human coronavirus 229E (HCoV-229E), human coronavirus OC43 (HCoV-OC43), human coronavirus NL63 (HCoV-NL63), or human coronavirus HKU1 (HCoV-HKU1).

33.-39. (canceled)

40. The method of claim 21, wherein the composition further comprises a pharmaceutically acceptable carrier, excipient, or diluent.

41.-54. (canceled)
